(19) United States
(12) Patent Application Publication
(10) Pub. No.: US 2025/0327058 A1
(43) Pub. Date: Oct. 23, 2025

(54) ENGINEERED PHENYLALANINE AMMONIA LYASE AND TYROSINE AMMONIA LYASE ENZYMES FOR PRODUCING AROMATIC COMPOUNDS

(71) Applicant: Ginkgo Bioworks, Inc., Boston, MA (US)

(72) Inventors: Nadia Parachin, Lexington, MA (US); Pichet Praveschotinunt, Medford, MA (US); Nathan W. Schmidt, Milton, MA (US)

(73) Assignee: Ginkgo Bioworks, Inc., Boston, MA (US)

(21) Appl. No.: 18/868,926

(22) PCT Filed: May 25, 2023
(86) PCT No.: PCT/US2023/067497

§ 371 Date: Nov. 25, 2024

Related U.S. Application Data
(60) Provisional application No. 63/346,101, filed on May 26, 2022.

(51) Int. Cl. C12N 9/00; C12N 1/18; C12N 1/20; C12N 9/02; C12N 9/88; C12P 7/42; C12R 1/19; C12R 1/84; C12R 1/865

(52) U.S. Cl. CPC C12N 9/93; C12N 1/18; C12N 1/20; C12N 9/0071; C12N 9/88; C12P 7/42; C12Y 114/14; C12Y 403/01023; C12Y 403/01024; C12Y 602/01012; C12R 2001/19; C12R 2001/84; C12R 2001/865

(57) ABSTRACT

Aspects of the disclosure relate to aromatic amino acid ammonia lyases (ALs), phenylalanine ammonia lyases (PALs), and tyrosine ammonia lyase (TALs), including engineered enzymes, and their use in catalyzing chemical reactions.

### CROSS-REFERENCE TO RELATED APPLICATIONS

This application claims the benefit under 35 U.S.C. § 119 (e) of U.S. Provisional Application No. 63/346,101, filed May 26, 2022, entitled, “ENGINEERED PHENYLALANINE AMMONIA LYASE AND TYROSINE AMMONIA LYASE ENZYMES FOR PRODUCING AROMATIC COMPOUNDS,” the entire disclosure of which is hereby incorporated by reference in its entirety.

### REFERENCE TO AN ELECTRONIC SEQUENCE LISTING

The contents of the electronic sequence listing (G091970083WO00-SEQ-KVC.xml; Size: 1,439,434 bytes; and Date of Creation: May 11, 2023) is herein incorporated by reference in its entirety.

### FIELD OF THE INVENTION

The present disclosure relates to the use of engineered phenylalanine ammonia lyase enzymes and tyrosine ammonia lyase enzymes for production of aromatic compounds.

### BACKGROUND

Aromatic compounds have useful pharmacological properties as well as properties useful for the flavor and fragrance industry. Trans-cinnamic acid can be used for producing flavors, dyes and pharmaceuticals. p-coumaric acid is a precursor of many phenolic compounds and its conjugates are of interest due to their antioxidant, anti-cancer, antimicrobial, antivirus, anti-inflammatory, antiplatelet aggregation, anxiolytic, antipyretic, analgesic, and anti-arthritis properties. Trans-cinnamic acid and p-coumaric acid are also highly sought after in the flavor and fragrance industries due their desirable characteristics. For example, trans-cinnamic acid has a honey-like odor and can be used to impart cinnamon-like flavors, while p-coumaric acid is found in many natural foods and beverages. Chemical synthesis of trans-cinnamic acid and p-coumaric acid is laborious and often results in low yields.

### SUMMARY

Aspects of the present disclosure relate to a host cell that comprises a heterologous polynucleotide encoding an aromatic amino acid ammonia lyase (AL), wherein the amino acid sequence of the AL comprises: a histidine (H) at a position corresponding to position 102 in the sequence of SEQ ID NO: 1; an isoleucine (I) at a position corresponding to position 104 in the sequence of SEQ ID NO: 1; a valine (V) at a position corresponding to position 104 in the sequence of SEQ ID NO: 1; a histidine (H) at a position corresponding to position 107 in the sequence of SEQ ID NO: 1; a serine(S) at a position corresponding to position 107 in the sequence of SEQ ID NO: 1; a tyrosine (Y) at a position corresponding to position 107 in the sequence of SEQ ID NO: 1; a methionine (M) at a position corresponding to position 108 in the sequence of SEQ ID NO: 1; or any combination thereof.

In some embodiments, the AL is a phenylalanine ammonia lyase (PAL). In some embodiments, the amino acid sequence of the PAL comprises substitutions at positions corresponding to the following positions in the sequence of SEQ ID NO: 1: positions 102, 104, and 218; positions 104, 108, and 218; positions 102, 104, 108, 218, and 222; positions 102 and 222; positions 102, 104, and 219; positions 102, 108, and 222; positions 102, 108, 218, and 222; positions 102 and 218; positions 102, 104, 108, and 222; positions 102, 104, and 108; positions 102, 218, and 222; positions 102, 104, 219, and 222; positions 102 and 108; positions 104 and 222; positions 102, 108, and 218; or positions 104 and 108.

In some embodiments, the amino acid sequence of the PAL comprises the following amino acid substitutions relative to the sequence of SEQ ID NO: 1: T102H, L104M, and G218A; L104M, L108T, and G218A; T102E, L104M, L108T, G218A, and M222L; T102S and M222L; T102H, L104M, and L219I; T102H, L104M, L108T, G218A, and M222V; T102S, L108T, and M222L; T102S, L108T, G218S, and M222L; T102E, L108T, and M222I; T102E and G218S; T102K, L104I, L108T, and M222L; T102S, L104M, and L108M; T102K, G218A, and M222T; T102S, L104M, L219I, and M222L; T102H and L108T; L104M and M222V; T102H, L104M, G218A, and M222T; T102S, L108V, and G218A; L104A, L108T, and G218A; L104V and L108T; or T102K, L108V, and M222L.

In some embodiments, the amino acid sequence of the PAL comprises the following amino acid substitutions relative to the sequence of SEQ ID NO: 1: T102H, L104A, and G218A; T102K, L104V, L219I, and M222V; T102K, L108V, and M222L; T102H, L108M, G218A, and M222T; T102K, L104A, and M222I; T102K and M222T; T102K and L104I; L104M and M222V; T102S, L108M, and G218S; T102E and L108M; T102E, L108M, and G218A; T102S and L108M; L102K and L108M; or L108M.

In some embodiments, the AL is a tyrosine ammonia lyase (TAL). In some embodiments, the amino acid sequence of the TAL comprises substitutions at positions corresponding to the following positions in the sequence of SEQ ID NO: 1: positions 104, 108, 219, and 222; positions 102, 108, 218, and 219; positions 102, 104, 108, 219, and 222; positions 102, 107, 108, 218, 219, and 222; positions 104, 108, 218, 219, and 222; positions 102, 104, 107, and 222; positions 102, 104, 107, 108, 219, and 222; positions 104, 218, and 222; positions 102, 108, 218, 219, and 222; positions 104, 108, and 218; positions 102, 107, 108, 219, and 222; positions 104, 107, 108, and 222; positions 102, 104, 108, 218, and 219; positions 102, 104, 107, 219, and 222; positions 102, 108, 218, and 222; positions 102, 108, and 222; positions 102, 104, 108, and 219; positions 102, 104, 107, 108, 218, 219, and 222; positions 102, 104, 107, 108, 218, and 219; positions 102, 107, 108, 219, and 222; positions 102, 104, 107, 108, 218, and 222; or positions 102, 104, 107, 108, and 219.

In some embodiments, the amino acid sequence of the TAL comprises the following amino acid substitutions relative to the sequence of SEQ ID NO: 1: L104A, L108Q, L219I, and M222N; T102S, L108Q, G218A, and L219I; T102H, L104M, L108M, L219I, and M222L; T102E, F107Y, L108M, G218S, L219I, and M222N; L104I, L108H, G218A, L219I, and M222V; T102E, L104M, F107Y, and M222I; T102E, L104V, F107Y, L108M, L219I, and M222T; T102S, L104I, G218S, L219I, and M222V; L104V, G218A, and M222L; T102K, L108H, G218A, L219I, and M222T; L104I, L108M, and G218S; T102H, F107Y, L108M, L219I, and M222V; L104V, F107H, L108Q, and M222L; T102K, L104A, L108Q, G218A, and L219I; T102S, L104A, F107S, L219I, and M222N; T102S, L108H, G218S, and M222V; T102K, L104A, L108H, L219I, and M222N; T102S, L108H, and M222N; T102H, L104M, L108M, and L219I; T102K, L104A, F107Y, L108V, G218A, L219I, and M222N; T102H, L108M, G218S, and M222L; T102E, L104M, F107Y, L108M, G218A, and L219I; T102E, L104V, F107H, and M222N; T102H, F107H, L108M, L219I, and M222T; T102H, L104V, F107S, L108Q, G218S, and M222T; T102E, L104M, F107S, L108M, G218A, and L219I; or T102E, L104V, F107Y, L108M, and L219I.

Aspects of the present disclosure relate to a host cell that comprises a heterologous polynucleotide encoding an AL, wherein the amino acid sequence of the AL comprises an amino acid substitution at a position corresponding to amino acid residue F107 relative to the sequence of SEQ ID NO: 1. In some embodiments, the amino acid sequence of the AL comprises: a histidine (H) at a position corresponding to position 107 in the sequence of SEQ ID NO: 1; a serine(S) at a position corresponding to position 107 in the sequence of SEQ ID NO: 1; or a tyrosine (Y) at a position corresponding to position 107 in the sequence of SEQ ID NO: 1.

Aspects of the present disclosure relate to a host cell that comprises: a first heterologous polynucleotide encoding an AL, wherein the amino acid sequence of the AL comprises one or more amino acid substitutions relative to the sequence of SEQ ID NO:1, and a second heterologous polynucleotide encoding a coumarate ligase (4CL).

Aspects of the present disclosure relate to a mixture comprising: a host cell comprising a first heterologous polynucleotide encoding an AL, wherein the amino acid sequence of the AL comprises one or more amino acid substitutions relative to the sequence of SEQ ID NO: 1, and a medium comprising exogenously supplied glucose, phosphoenolpyruvate, erythrose 4-phosphate, 3-deoxy-D-arabino-hept-2-ulosonate 7-phosphate, 3-dehydroquinate, 3-dehydroshikimate, shikimate, chorismate, prephenate, phenylpyruvate, hydroxyphenylpyruvate, phenylalanine, or tyrosine.

In some embodiments, the amino acid sequence of the AL comprises an amino acid substitution at a position corresponding to amino acid residue 102, 104, 107, 108, 218, 219, or 222 relative to the sequence of SEQ ID NO: 1. In some embodiments, the AL comprises: a serine(S) at a position corresponding to position 102 in the sequence of SEQ ID NO: 1; a glutamic acid (E) at a position corresponding to position 102 in the sequence of SEQ ID NO: 1; a lysine (K) at a position corresponding to position 102 in the sequence of SEQ ID NO: 1; a histidine (H) at a position corresponding to position 102 in the sequence of SEQ ID NO: 1; a methionine (M) at a position corresponding to position 104 in the sequence of SEQ ID NO: 1; an alanine (A) at a position corresponding to position 104 in the sequence of SEQ ID NO: 1; an isoleucine (I) at a position corresponding to position 104 in the sequence of SEQ ID NO: 1; a valine (V) at a position corresponding to position 104 in the sequence of SEQ ID NO: 1; a histidine (H) at a position corresponding to position 107 in the sequence of SEQ ID NO: 1; a serine(S) at a position corresponding to position 107 in the sequence of SEQ ID NO: 1; a tyrosine (Y) at a position corresponding to position 107 in the sequence of SEQ ID NO: 1; a threonine (T) at a position corresponding to position 108 in the sequence of SEQ ID NO: 1; a valine (V) at a position corresponding to position 108 in the sequence of SEQ ID NO: 1; a glutamine (Q) at a position corresponding to position 108 in the sequence of SEQ ID NO: 1; a methionine (M) at a position corresponding to position 108 in the sequence of SEQ ID NO: 1; an alanine (A) at a position corresponding to position 218 in the sequence of SEQ ID NO: 1; a serine(S) at a position corresponding to position 218 in the sequence of SEQ ID NO: 1; an isoleucine (I) at a position corresponding to position 219 in the sequence of SEQ ID NO: 1; a leucine (L) at a position corresponding to position 222 in the sequence of SEQ ID NO: 1; an asparagine (N) at a position corresponding to position 222 in the sequence of SEQ ID NO: 1; an isoleucine (I) at a position corresponding to position 222 in the sequence of SEQ ID NO: 1; a valine (V) at a position corresponding to position 222 in the sequence of SEQ ID NO: 1; a threonine (T) at a position corresponding to position 222 in the sequence of SEQ ID NO: 1; or any combination thereof.

In some embodiments, the AL is a PAL. In some embodiments, relative to the sequence of SEQ ID NO: 1, the PAL comprises substitutions at positions corresponding to the following positions in the sequence of SEQ ID NO: 1: positions 102, 104, and 218; positions 104, 108, and 218; positions 102, 104, 108, 218, and 222; positions 102 and 222; positions 102, 104, and 219; positions 102, 108, and 222; positions 102, 108, 218, and 222; positions 102 and 218; positions 102, 104, 108, and 222; positions 102, 104, and 108; positions 102, 218, and 222; positions 102, 104, 219, and 222; positions 102 and 108; positions 104 and 222; positions 102, 108, and 218; or positions 104 and 108.

In some embodiments, the amino acid sequence of the PAL comprises the following amino acid substitutions relative to the sequence of SEQ ID NO: 1: T102H, L104M, and G218A; L104M, L108T, and G218A; T102E, L104M, L108T, G218A, and M222L; T102S and M222L; T102H, L104M, and L219I; T102H, L104M, L108T, G218A, and M222V; T102K and G218A; T102S, L108T, and M222L; T102S, L108T, G218S, and M222L; T102E, L108T, and M222I; T102E and G218S; T102K, L104I, L108T, and M222L; T102S, L104M, and L108M; T102K, G218A, and M222T; T102S, L104M, L219I, and M222L; T102H and L108T; L104M and M222V; T102H, L104M, G218A, and M222T; T102S, L108V, and G218A; L104A, L108T, and G218A; L104V and L108T; or T102K, L108V, and M222L.

In some embodiments, the AL is a TAL. In some embodiments, the TAL comprises substitutions at positions corresponding to the following positions in the sequence of SEQ ID NO: 1: positions 104, 108, 219, and 222; positions 102, 108, 218, and 219; positions 102, 104, 108, 219, and 222; positions 102, 107, 108, 218, 219, and 222; positions 104, 108, 218, 219, and 222; positions 102, 104, 107, and 222; positions 102, 104, 107, 108, 219, and 222; positions 104, 218, and 222; positions 102, 108, 218, 219, and 222; positions 104, 108, and 218; positions 102, 107, 108, 219, and 222; positions 104, 107, 108, and 222; positions 102, 104, 108, 218, and 219; positions 102, 104, 107, 219, and 222; positions 102, 108, 218, and 222; positions 102, 108, and 222; positions 102, 104, 108, and 219; positions 102, 104, 107, 108, 218, 219, and 222; positions 102, 104, 107, 108, 218, and 219; positions 102, 107, 108, 219, and 222; positions 102, 104, 107, 108, 218, and 222; or positions 102, 104, 107, 108, and 219.

In some embodiments, the amino acid sequence of the TAL comprises the following amino acid substitutions relative to the sequence of SEQ ID NO: 1: L104A, L108Q, L219I, and M222N; T102S, L108Q, G218A, and L219I; T102H, L104M, L108M, L219I, and M222L; T102E, F107Y, L108M, G218S, L219I, and M222N; L104I, L108H, G218A, L219I, and M222V; T102E, L104M, F107Y, and M222I; T102E, L104V, F107Y, L108M, L219I, and M222T; T102S, L104I, G218S, L219I, and M222V; L104V, G218A, and M222L; T102K, L108H, G218A, L219I, and M222T; L104I, L108M, and G218S; T102H, F107Y, L108M, L219I, and M222V; L104V, F107H, L108Q, and M222L; T102K, L104A, L108Q, G218A, and L219I; T102S, L104A, F107S, L219I, and M222N; T102S, L108H, G218S, and M222V; T102K, L104A, L108H, L219I, and M222N; T102S, L108H, and M222N; T102H, L104M, L108M, and L219I; T102K, L104A, F107Y, L108V, G218A, L219I, and M222N; T102H, L108M, G218S, and M222L; T102E, L104M, F107Y, L108M, G218A, and L219I; T102E, L104V, F107H, and M222N; T102H, F107H, L108M, L219I, and M222T; T102H, L104V, F107S, L108Q, G218S, and M222T; T102E, L104M, F107S, L108M, G218A, and L219I; or T102E, L104V, F107Y, L108M, and L219I.

In some embodiments, the amino acid sequence of the TAL comprises the following amino acid substitutions relative to the sequence of SEQ ID NO: 1: T102E, L104V, F107Y, and L108H; T102E, F107Y, L108H, G218A, and M222I; T102S, F107Y, L108H, G218A, and M222T; T102E, L104M, F107Y, L108H, and G218A; L219I and M222T; F107Y, L108H, L219I, and M222T; L104A, L108Q, L219I, and M222N; T102S, L108Q, G218A, and L219I; T102H, L104M, L108M, and L219I; M222L; T102E, F107Y, L108M, and G218S; L219I and M222N; L104I, L108H, G218A, and L219I; M222V; T102E, L104M, F107Y, and M222I; T102E, F107Y, L108H, and M222I; T102E, F107Y, L108H, and G218A; T102S, F107Y, and L108H; T102E, F107Y, L108H, and M222T; or T102E, F107Y, L108H, and L219I.

In some embodiments, the AL comprises an amino acid sequence that has at least 90% identity to the sequence of SEQ ID NO: 1. In some embodiments, the heterologous polynucleotide comprises a sequence that is at least 90% identical to the sequence of SEQ ID NO: 2. In some embodiments, the host cell is a bacterial cell, an archaebacterial cell, an algal cell, a fungal cell, a yeast cell, a plant cell, an animal cell, a mammalian cell, or a human cell. In some embodiments, the host cell is a filamentous fungi cell or a yeast cell. In some embodiments, the yeast cell is a Saccharomyces cell, a Yarrowia cell, a Komagataella cell, or a Pichia cell. In some embodiments, the Saccharomyces cell is a Saccharomyces cerevisiae cell. In some embodiments, the yeast cell is Yarrowia cell. In some embodiments, the host cell is a bacterial cell. In some embodiments, the bacterial cell is an E. coli cell.

In some embodiments, the AL is able to convert phenylalanine to trans-cinnamic acid. In some embodiments, the AL is able to convert tyrosine to p-coumaric acid. In some embodiments, the host cell comprises one or more enzymes of the shikimate pathway capable of converting phosphoenolpyruvate and erythrose 4-phosphate to chorismate. In some embodiments, one or more of the enzymes of the shikimate pathway are encoded by a heterologous polynucleotide. In some embodiments, the amino acid sequence(s) of one or more of the enzymes of the shikimate pathway comprise one or more substitutions relative to the amino acid sequence(s) of a wild-type shikimate pathway enzyme. In some embodiments, the host cell further comprises a heterologous polynucleotide encoding a cinnamate 4-hydroxylase (C4H), a heterologous polynucleotide encoding a coumarate ligase (4CL), or both. In some embodiments, the amino acid sequence of C4H comprises one or more substitutions relative to the amino acid sequence of a parent C4H (SEQ ID NO: 389). In some embodiments, the amino acid sequence of 4CL comprises one or more substitutions relative to the amino acid sequence of wild-type 4CL. In some embodiments, the host cell further comprises a heterologous polynucleotide encoding one, two, three, four, five, or all of: a coumarate ligase (4CL), a double bond reductase (DBR), a chalcone synthase (CHS), a chalcone 3-hydroxylase (CH3H), an O-methyltransferase (OMT), and an UDP dependent glycosyltransferase (UGT). In some embodiments, the amino acid sequence(s) of one, two, three, four, five, or all of 4CL, DBR, CHS, CH3H, OMT, or UGT comprises one or more substitutions relative to the amino acid sequence(s) of a wild-type version of the protein.

Aspects of the present disclosure relate to an AL, wherein the amino acid sequence of the AL comprises: a histidine (H) at a position corresponding to position 102 in the sequence of SEQ ID NO: 1; an isoleucine (I) at a position corresponding to position 104 in the sequence of SEQ ID NO: 1; a valine (V) at a position corresponding to position 104 in the sequence of SEQ ID NO: 1; a histidine (H) at a position corresponding to position 107 in the sequence of SEQ ID NO: 1; a serine(S) at a position corresponding to position 107 in the sequence of SEQ ID NO: 1; a tyrosine (Y) at a position corresponding to position 107 in the sequence of SEQ ID NO: 1; a methionine (M) at a position corresponding to position 108 in the sequence of SEQ ID NO: 1; or any combination thereof.

In some embodiments, the AL is a PAL. In some embodiments, the amino acid sequence of the AL comprises substitutions at positions corresponding to the following positions in the sequence of SEQ ID NO: 1: positions 102, 104, and 218; positions 104, 108, and 218; positions 102, 104, 108, 218, and 222; positions 102 and 222; positions 102, 104, and 219; positions 102, 108, and 222; positions 102, 108, 218, and 222; positions 102 and 218; positions 102, 104, 108, and 222; positions 102, 104, and 108; positions 102, 218, and 222; positions 102, 104, 219, and 222; positions 102 and 108; positions 104 and 222; positions 102, 108, and 218; or positions 104 and 108.

In some embodiments, the amino acid sequence of the AL comprises the following amino acid substitutions relative to the sequence of SEQ ID NO: 1: T102H, L104M, and G218A; L104M, L108T, and G218A; T102E, L104M, L108T, G218A, and M222L; T102S and M222L; T102H, L104M, and L219I; T102H, L104M, L108T, G218A, and M222V; T102S, L108T, and M222L; T102S, L108T, G218S, and M222L; T102E, L108T, and M222I; T102E and G218S; T102K, L104I, L108T, and M222L; T102S, L104M, and L108M; T102K, G218A, and M222T; T102S, L104M, L219I, and M222L; T102H and L108T; L104M and M222V; T102H, L104M, G218A, and M222T; T102S, L108V, and G218A; L104A, L108T, and G218A; L104V and L108T; or T102K, L108V, and M222L.

In some embodiments, the amino acid sequence of the AL comprises the following amino acid substitutions relative to the sequence of SEQ ID NO: 1: T102H, L104A, and G218A; T102K, L104V, L219I, and M222V; T102K, L108V, and M222L; T102H, L108M, G218A, and M222T; T102K, L104A, and M222I; T102K and M222T; T102K and L104I; L104M and M222V; T102S, L108M, and G218S; T102E and L108M; T102E, L108M, and G218A; T102S and L108M; L102K and L108M; or L108M.

In some embodiments, the AL is a TAL. In some embodiments, the amino acid sequence of the AL comprises substitutions at positions corresponding to the following positions in the sequence of SEQ ID NO: 1: positions 104, 108, 219, and 222; positions 102, 108, 218, and 219; positions 102, 104, 108, 219, and 222; positions 102, 107, 108, 218, 219, and 222; positions 104, 108, 218, 219, and 222; positions 102, 104, 107, and 222; positions 102, 104, 107, 108, 219, and 222; positions 104, 218, and 222; positions 102, 108, 218, 219, and 222; positions 104, 108, and 218; positions 102, 107, 108, 219, and 222; positions 104, 107, 108, and 222; positions 102, 104, 108, 218, and 219; positions 102, 104, 107, 219, and 222; positions 102, 108, 218, and 222; positions 102, 108, and 222; positions 102, 104, 108, and 219; positions 102, 104, 107, 108, 218, 219, and 222; positions 102, 104, 107, 108, 218, and 219; positions 102, 107, 108, 219, and 222; positions 102, 104, 107, 108, 218, and 222; or positions 102, 104, 107, 108, and 219.

In some embodiments, the amino acid sequence of the AL comprises the following amino acid substitutions relative to the sequence of SEQ ID NO: 1: L104A, L108Q, L219I, and M222N; T102S, L108Q, G218A, and L219I; T102H, L104M, L108M, L219I, and M222L; T102E, F107Y, L108M, G218S, L219I, and M222N; L104I, L108H, G218A, L219I, and M222V; T102E, L104M, F107Y, and M222I; T102E, L104V, F107Y, L108M, L219I, and M222T; T102S, L104I, G218S, L219I, and M222V; L104V, G218A, and M222L; T102K, L108H, G218A, L219I, and M222T; L104I, L108M, and G218S; T102H, F107Y, L108M, L219I, and M222V; L104V, F107H, L108Q, and M222L; T102K, L104A, L108Q, G218A, and L219I; T102S, L104A, F107S, L219I, and M222N; T102S, L108H, G218S, and M222V; T102K, L104A, L108H, L219I, and M222N; T102S, L108H, and M222N; T102H, L104M, L108M, and L219I; T102K, L104A, F107Y, L108V, G218A, L219I, and M222N; T102H, L108M, G218S, and M222L; T102E, L104M, F107Y, L108M, G218A, and L219I; T102E, L104V, F107H, and M222N; T102H, F107H, L108M, L219I, and M222T; T102H, L104V, F107S, L108Q, G218S, and M222T; T102E, L104M, F107S, L108M, G218A, and L219I; or T102E, L104V, F107Y, L108M, and L219I. In some embodiments, the amino acid sequence of the AL comprises an amino acid sequence that has at least 90% identity to the sequence of SEQ ID NO: 1.

Aspects of the present disclosure relate to an AL, wherein the amino acid sequence of the AL comprises an amino acid substitution at a position corresponding to amino acid residue F107 relative to the sequence of SEQ ID NO: 1. In some embodiments, the amino acid sequence of the AL comprises: a histidine (H) at a position corresponding to position 107 in the sequence of SEQ ID NO: 1; a serine(S) at a position corresponding to position 107 in the sequence of SEQ ID NO: 1; a tyrosine (Y) at a position corresponding to position 107 in the sequence of SEQ ID NO: 1. In some embodiments, the amino acid sequence of the AL comprises an amino acid substitution at a position corresponding to amino acid residue 102, 104, 108, 218, 219, or 222 relative to the sequence of SEQ ID NO: 1.

In some embodiments, the AL produces more trans-cinnamic acid per unit time than an AL with an amino acid sequence comprising the sequence of SEQ ID NO: 1. In some embodiments, the AL can produce at least 10%, 20%, 30%, 40%, 50%, 60%, 70%, 80%, 90%, 100%, 200%, or 300% more trans-cinnamic acid per unit time than a AL with an amino acid sequence comprising the sequence of SEQ ID NO: 1. In some embodiments, the AL can produce at least 10%, 20%, 30%, 40%, 50%, 60%, 70%, 80%, 90%, 100%, 200%, or 300% more trans-cinnamic acid per unit time than coumarate per unit time.

In some embodiments, the AL produces more coumarate per unit time than a TAL with an amino acid sequence comprising the sequence of SEQ ID NO: 1. In some embodiments, the AL can produce at least 10%, 20%, 30%, 40%, 50%, 60%, 70%, 80%, 90%, 100%, 200%, or 300% more coumarate per unit time than a TAL with an amino acid sequence comprising the sequence of SEQ ID NO: 1. In some embodiments, the AL can produce at least 10%, 20%, 30%, 40%, 50%, 60%, 70%, 80%, 90%, 100%, 200%, or 300% more coumarate per unit time than trans-cinnamic acid per unit time.

Aspects of the present disclosure relate to a method of producing an aromatic compound, comprising contacting phenylalanine and/or tyrosine with any host cell of the present disclosure or any AL of the present disclosure. In some embodiments, the method comprises contacting phenylalanine. In some embodiments, the method comprises contacting tyrosine.

In some embodiments, the aromatic compound is a flavor or fragrance compound. In some embodiments, the aromatic compound is a phenylpropanoid. In some embodiments, the aromatic compound is a sweetener. In some embodiments, the aromatic compound is a flavonoid. In some embodiments, the aromatic compound is a flavanone. In some embodiments, the aromatic compound is eriodictyol or a glycoside and/or alkoxy derivative thereof. In some embodiments, the aromatic compound is hesperetin. In some embodiments, the aromatic compound is a dihydrochalcone. In some embodiments, the aromatic compound is hesperetin dihydrochalcone 4′-O-glucoside (HDG). In some embodiments, the aromatic compound is vanillin. In some embodiments, the aromatic compound is an hydroxycinnamic acid or a derivative thereof. In some embodiments, the hydroxycinnamic acid or the derivative thereof is coumaric acid, ferulic acid, sinapic acid, caffeic acid, chlorogenic acid, or rosmarinic acid. In some embodiments, the aromatic compound is ferulic acid.

Aspects of the present disclosure relate to a method of improving an aromatic compound manufacturing mixture, comprising contacting the mixture with any of the ALs described in the present disclosure. In some embodiments, the method is a method of improving a flavor or fragrance manufacturing mixture. In some embodiments, the aromatic compound manufacturing mixture comprises a shikimate pathway product. In some embodiments, the shikimate pathway product comprises: chorismate, prephenate, phenylpyruvate, hydroxyphenylpyruvate, phenylalanine, or tyrosine. In some embodiments, improving comprises converting phenylalanine to trans-cinnamic acid. In some embodiments, improving comprises converting tyrosine to coumarate. In some embodiments, improving comprises promoting production of an aromatic compound. In some embodiments, the method occurs in vitro.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used in this disclosure is for the purpose of description and should not be regarded as limiting. The use of “including,” “comprising,” or “having,” “containing,” “involving,” and variations of thereof in this disclosure, is meant to encompass the items listed thereafter and equivalents thereof as well as additional items. As used in this specification and the appended claims, the singular forms “a,” “an” and “the” include plural referents unless the content clearly dictates otherwise.

### BRIEF DESCRIPTION OF THE DRAWINGS

The following drawings form part of the present specification and are included to further demonstrate certain aspects of the present disclosure, which may be better understood by reference to one or more of these drawings in combination with the detailed description of specific embodiments presented in this disclosure. The accompanying drawings are not intended to be drawn to scale. The drawings are illustrative only and are not required for enablement of the disclosure. For purposes of clarity, not every component may be labeled in every drawing. In the drawings:

FIG. 1 is a schematic showing the metabolic pathway upstream of the PAL and TAL substrates described herein.

FIG. 2 is a schematic showing the reaction catalyzed by PAL and TAL enzymes.

FIG. 3 is a graph showing data from a secondary screen described in Example 1 of strains expressing a protein engineering library containing variant PALs that included amino acid substitutions relative to the wild-type PAL from Anabaena variabilis (AvPAL; UniProKB Accession No. Q3M5Z3; SEQ ID NO: 1). A strain expressing wild-type AvPAL was included as a positive control. A strain expressing GFP was included as a negative control. The Y-axis shows the kinetic absorbance measurements collected at 290 nm per minute for each strain on the X-axis.

FIG. 4 is a graph showing data from a secondary screen described in Example 2 of a protein engineering library described in Example 1, screened for TAL activity. The Y-axis shows the whole cell assay tCA (mM) concentration normalized to the OD600 of the culture for each strain on the X-axis. The data show the plotting of biological triplicates. A strain expressing wild-type AvPAL was included as a positive control (called “avPAL positive control”). A strain expressing GFP was included as a negative control. A strain expressing RsTAL was also included as a positive control (called “rsTAL positive control”).

### DETAILED DESCRIPTION OF THE INVENTION

The present disclosure provides, in some aspects, engineered enzymes that are capable of enhanced aromatic amino acid processing, e.g., phenylalanine and/or tyrosine processing. These enzymes include phenylalanine ammonia lyases (PALs), which are phenylalanine converting enzymes that catalyze a reaction converting L-phenylalanine to ammonia and trans-cinnamic acid, tyrosine ammonia lyases (TALs), which are tyrosine converting enzymes that catalyze a reaction converting L-tyrosine to ammonia and p-coumaric acid, and enzymes capable of processing both phenylalanine and tyrosine. An enzyme that is capable of converting L-phenylalanine to ammonia and trans-cinnamic acid and/or converting L-tyrosine to ammonia and p-coumaric acid is referred to herein as an aromatic amino acid ammonia lyase (also referred to herein as an AL). In some embodiments, an AL is a PAL. In some embodiments, an AL is a TAL. In some embodiments, an AL is a PAL and a TAL. Accordingly, the disclosure provides, in some aspects, ALs, PALs, and TALs.

The disclosed enzymes and host cells comprising such enzymes may be used to promote reactions that use phenylalanine and/or tyrosine as substrates, e.g., to produce increased quantities of aromatic compounds including, for example, trans-cinnamic acid and/or p-coumaric acid, and may also be used in other industrial settings. For example, in the flavor and fragrance industries, aromatic compounds (e.g., trans-cinnamic acid and p-coumaric acid) are sought after due to their desirable flavor and fragrance characteristics. The disclosure is directed, in part, to the discovery of AL enzymes capable of processing phenylalanine and/or tyrosine to increase biosynthesis of trans-cinnamic acid and/or p-coumaric acid, nucleic acids encoding the same, and host cells capable of expressing AL enzymes, e.g., to produce increased quantities of trans-cinnamic acid and/or p-coumaric acid.

Aromatic Compounds Aspects of the disclosure are useful for the production of aromatic compounds. As used in this disclosure, the term “aromatic compound” refers to a compound that comprises a phenyl group. The aromatic compounds of this disclosure can be produced by enzymatic activity or metabolism from products of the shikimate pathway, e.g., aromatic compound precursors (e.g., chorismate and prephenate), and/or other aromatic compounds (e.g., coumarate), either in vitro or in vivo. Aromatic compounds have numerous clinical and industrial uses including production of antioxidants, cosmetics, perfumes, UV screens, and anticancer, anti-viral, anti-inflammatory, wound healing, and antibacterial agents. In some embodiments, an aromatic compound is a flavor or fragrance compound that can be produced by enzymatic activity or metabolism from products of the shikimate pathway.

Aromatic compounds include, but are not limited to: glucosinolates, coumarins, isothiocyanates, ubiquinons, lignins, lignans, stilbenoids, flavonoids (e.g., condensed tanins, proanthocyanides, or anthyocyanins), C6 aromatic-C2 compounds (e.g., 2-phenylethanol, phenylacetaldehyde, or phenylacetonitrile), benzeneoids (e.g., benzyl alcohol, methyl benzoate, or benzyl benzoate), phenylpropanoids (e.g., eugenol, methyl eugenol, chavicol, and isoeugenol), and any other polyphenolic compounds useful in flavor or fragrance applications. In some embodiments, the aromatic compound is a flavonoid. In some embodiments, the aromatic compound is a flavanone. In some embodiments, the aromatic compound is eriodictyol, homoeriodictyol, or sterubin, or a glycoside or alkoxy derivative of any thereof (e.g., eriocitrin). In some embodiments, an aromatic compound is naringenin, naringin, or hesperetin. In some embodiments, an aromatic compound is a hesperetin glycoside, e.g., hesperetin 7-O-glycoside (also known as hesperidin). In some embodiments, an aromatic compound comprises a dihydrochalcone group, e.g., a substituted dihydrochalcone, e.g., a hesperetin dihydrochalcone, e.g., neohesperidin dihydrochalcone or hesperetin dihydrochalcone. In some embodiments, the aromatic compound is a hesperetin dihydrochalcone O-glucoside (e.g., hesperetin dihydrochalcone 4′-O-glucoside (HDG)). In some embodiments, the aromatic compound is vanillin. In some embodiments, the aromatic compound is raspberry ketone. In some embodiments, the aromatic compound is methyl cinnamate. In some embodiments, the aromatic compound is naringin. In some embodiments, the aromatic compound is ferulic acid. In some embodiments, an aromatic compound is naturally occurring, e.g., is produced by a naturally occurring cell. In some embodiments, an aromatic compound is synthetic.

In some embodiments, an aromatic compound is a phenylpropanoid. As used in this disclosure, “phenylpropanoids” are compounds comprising an aromatic ring and (i) a three-carbon substituted or unsubstituted propene or substituted or unsubstituted propenylene tail, wherein the propene or propenylene tail is attached to the aromatic ring or (ii) a three-carbon substituted or unsubstituted propane or substituted or unsubstituted propanylene tail, wherein the propane or propanylene tail is attached to the aromatic ring. Non-limiting examples of phenylpropanoids include hydroxycinnamic acids and derivatives thereof, flavonoids, flavanones, and phenylpropanoid glycosides. In some embodiments, a phenylpropanoid is hesperetin, eriodictyol dihydrochalcone, hesperetin dihydrochalcone 4′-O-glucoside (HDG), trans-cinnamic acid, or coumarate.

In some embodiments, a phenylpropanoid is a hydroxycinnamic acid. Hydroxycinnamic acids are compounds that comprise an aromatic ring and a propenoic acid attached to the aromatic ring. Hydroxycinnamic acids are known to those of skill in the art and are generally composed of a carbon backbone that varies in length from C6 to C3 with a variety of substituents such as caffeic acid, chlorogenic acid, and quinic acid. These organic compounds are hydroxy derivatives of cinnamic acid. Non-limiting examples of hydroxycinnamic acids include m-coumaric acid, o-coumaric acid, p-coumaric acid, caffeic acid, ferulic acid, and sinapic acid. In some embodiments, a hydroxycinnamic acid derivative is an ester, amide, or hydrazide derivative of an hydroxycinnamic acid. For example, rosmarinic acid is an ester derivative of caffeic acid and chlorogenic acids are ester derivatives of hydroxycinnamic acids with quinic acid. In some embodiments, a chlorogenic acid is 3-caffeoylquinic acid.

In some embodiments, a hydroxycinnamic acid or derivative thereof is m-coumaric acid, o-coumaric acid, p-coumaric acid, caffeic acid, ferulic acid, sinapic acid, rosmarinic acid, or a chlorogenic acid.

In some embodiments, a hydroxycinnamic acid or a derivative thereof is a compound of Formula (1):

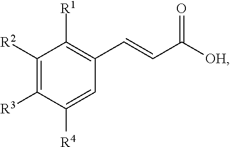

wherein:
R¹ is H, OH, OCH₃, CH₃, or OCH₂COOH;
R² is H, OH, OCH₃, CH₂CH═C(CH₃)₂, CO(CH₂)₂Ph, CH₂CH═C(CH₃)CH₂OH, COOH, 3,4-[—OCH₂O—], NH₂, Br, C(CH₃)₃, OCH₂COOH, NO₂, CH₃, or γ,γ-dimethylallyl;
R³ is H, OH, CH₂CH═C(CH₃)₂, CO(CH₂)₂Ph, CH₂CH═C(CH₃)CH₂OH, OCH₂COOH, N(CH₃)₂, OCH₃, CHO, NO₂, Cl, NH₂, SO₃H, CH₃, or Oac; and
R⁴ is H, OCH₃, Br, C(CH₃)₃, OH, or NO₂, provided that at least one of R¹—R⁴ is OH.

The abbreviation “Ph” represents a phenyl group.

In some embodiments, a hydroxycinnamic acid derivative is a compound of Formula (2):

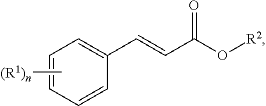

wherein:
R¹ is —OH, —OCH₃, or halogen;
R² is allyl, 1-naphthylmethyl, CH₂CH₂Ph, 3,4-dihydroxyphenethyl, 2-phenoxyethyl, 2-hydroxyethyl, tetradecyl, hexadecyl; octadecyl, hexylEt, CH₃, 3-phenylprop-2-en-1-yl, 4-allyl-2,6-dimethoxyphenyl, CH₂Ph; CH₂ CH₂CH(CH₃)₂, phenethyl, 2-(1-naftyl)-ethyl; 2-(2-naftyl)-ethyl, CH₂COOH, CH(CH₃) COOH, bornyl, i-Pr, or Bu; and
n is 1, 2, 3, 4, or 5.

The abbreviation “Et” represents an ethyl group.

The abbreviation “Pr” represents a propyl group.

The abbreviation “i-Pr” represents an isopropyl group.

The abbreviation “Bu” represents a butyl group.

In some embodiments, a hydroxycinnamic acid derivative is a compound of Formula (3):

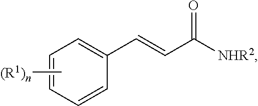

wherein:
R¹ is —OH, —OCH₃, i-Pr, —O-isopentenyl, geranyl, —O-geranyl, —NO₂, 3,4-(O—CH₂—O), or halogen;
R² is 2-(3-methoxy-4-hydroxyphenyl)-ethyl, 2-(4-hydroxyphenyl)-ethyl, hexyl, H, NH₃, 3-methylbut-2-enyl, OH, OMe, OEt, i-Pr, i-Bu, isopentyl, allyl, Ph, 2-OH-Ph, 3-OH-Ph, 4-OH-Ph, Bn, phenethyl, pyrollidinyl, piperidinyl, morpholinyl, (CH₃)₂, dopaminyl, N-(2-(4-hydroxyphenyl)ethyl)-N-methyl, 2-(3,4-dihydroxyphenyl)-ethyl, NH₂, 2-NO₂-Ph, 2,4-diNO₂-Ph, 2-Cl-Ph, 3-Cl-Ph, 4-Cl-Ph, 4-OMe-Ph, 2-CH₃-Ph, N(CH₃)₂, N(Et)₂, N(C₂H₄OH)₂, i-PrNH, n-Bu, NHNH₂, NHCOPh, NHCOPy, 2-(N-acetylamino)-ethyl, NH-(pyridine-2-yl), NH(CH₂)₂-(indole-3-yl), NHR²: Gly; Ala; Val; Phe; Tyr; or 3′,4′-diOH-Phe, NHR²: Gly; or Val, NHR²: L-Val-OMe; L-Leu-OMe; L-Phe-t-Bu; L-Tyr-OMe; or L-Phe (4-F-Ph)-Me, or NHR²: L-Tyr-OMe; L-Phe (4-F-Ph)-Me; or L-Phe-t-Bu. See also, e.g., Sova et al., Mini Rev Med Chem. 2012 July; 12(8):749-67; and
n is 1, 2, 3, 4, or 5.

The abbreviation “Me” represents a methyl group.

The abbreviation “Bn” represents a benzyl group.

Hydroxycinnamic acids and their derivatives have numerous clinical and industrial applications including use in production of flavoring agents, fragrances, antioxidants, antivirals, antibacterials, and antifungals. As a non-limiting example, hydroxycinnamic acids, including caffeic, ferulic, and chlorogenic acid have been shown to have antioxidant properties and can act as superoxide anion scavengers. Chlorogenic acids have also been used as antioxidants and anti-inflammatory compounds for treatment of numerous diseases including cardiovascular disease, type 2 diabetes and Alzheimer's disease. Cinnamates, which are hydroxycinnamic acid derivatives, have also been found to contribute to the antioxidative effects of white wine. Trans-cinnamic acid can be used for producing flavors, dyes and pharmaceuticals. p-coumaric acid is a precursor of many phenolic compounds and its conjugates are of interest due to their antioxidant, anti-cancer, antimicrobial, antivirus, anti-inflammatory, antiplatelet aggregation, anxiolytic, antipyretic, analgesic, and anti-arthritis properties. See also, e.g., Sova et al., Mini Rev Med Chem. 2012 July; 12(8):749-67.

### Phenylalanine Ammonia Lyases (PALs)

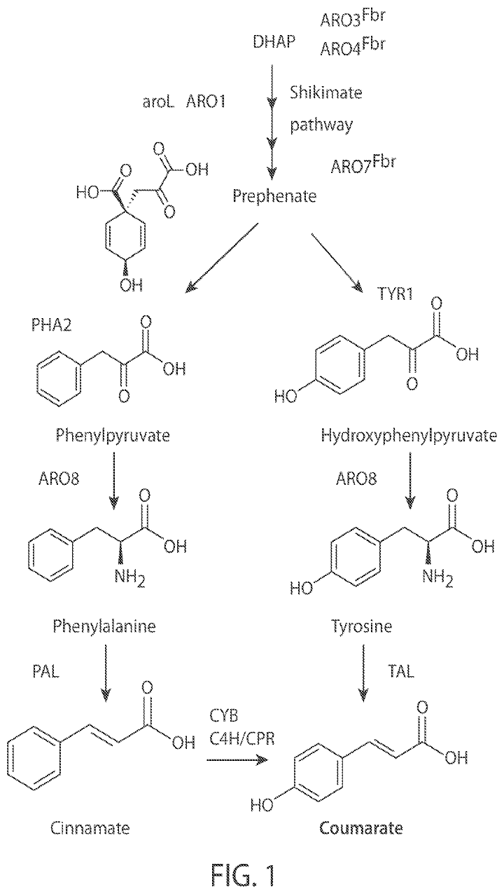

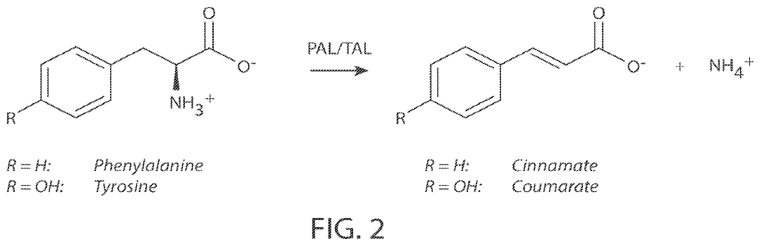

In some embodiments, an AL is a PAL (i.e., it is an enzyme capable of converting L-phenylalanine to ammonia and trans-cinnamic acid). As used in this disclosure, a “phenylalanine ammonia lyase” or “(PAL)” refers to an enzyme that catalyzes the conversion of L-phenylalanine to ammonia and trans-cinnamic acid (FIG. 2). In some embodiments, a PAL is a L-phenylalanine converting enzyme. Naturally occurring PALs, along with tyrosine ammonia lyases (TALs), and histidine ammonia lyases (HALs), are members of the aromatic amino acid lyase family of enzymes. Such enzymes are characterized by the presence of a co-factor (4-methyldiene-imidazol-5-one (MIO)) in their active sites, formed in naturally occurring PALs by autocatalytic cyclization and dehydration of an internal tri-peptide segment (e.g., an Ala-Ser-Gly). PALs are found in a variety of microorganisms (e.g., cyanobacteria, bacteria (e.g., actinobacteria), and extremophiles), fungi (e.g., yeast), plants, and protists (e.g., algae), and are central to the phenylpropanoid pathway of plants, but do not naturally occur in mammalian animals such as humans. The phenylpropanoid pathway transforms aromatic amino acids produced from carbon sources in the shikimate pathway into a variety of different aromatic compounds. Naturally occurring PALs produce trans-cinnamic acid from L-phenylalanine, which can then be further processed by downstream enzymes such as, e.g., cinnamate 4-hydroxylase, 4-coumarate-coenzyme A ligase, chalcone synthase, or flavonol synthase (FIG. 1). Naturally occurring PALs can have different substrate and/or product specificities; for example, PALs from dicotyledonous plants predominantly deaminate L-phenylalanine to ammonia and trans-cinnamic acid, whereas PALs from yeast and some monocot plants (e.g., maize) are known to convert L-phenylalanine and L-tyrosine to trans-cinnamic acid and p-coumaric acid, respectively. In a given plant species, multiple PAL-encoding genes may be found, increasing the number of naturally occurring PAL isoforms available for engineering. PAL enzymes occur as tetramers, with naturally occurring tetramers having molecular weights of about 64-478 kDa; heterotetramers of different naturally occurring PAL isoforms have been observed.

An AL of the disclosure that is a PAL can use L-phenylalanine as a substrate. In some embodiments, an AL, e.g., a PAL, exhibits specificity for L-phenylalanine compared to other amino acids (e.g., compared to L-tyrosine or L-histidine). In some embodiments, a PAL produces ammonia and trans-cinnamic acid from L-phenylalanine. In some embodiments, an AL, e.g., a PAL, predominantly consumes L-phenylalanine relative to one or more other amino acids; e.g., may consume L-phenylalanine at a rate at least 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, or 6-fold higher (e.g., 2-fold to 6-fold more) relative to one or more other amino acids (e.g., relative to L-tyrosine or L-histidine). In some embodiments, an AL can convert L-tyrosine into ammonia and p-coumaric acid. In some embodiments, an AL can convert L-histidine into ammonia and urocanic acid.

In some embodiments, an AL (e.g., a PAL) comprises aromatic, alkyl, and/or hydrophobic amino acids at one or both positions corresponding to position 107 and/or 108 in SEQ ID NO: 1. In some embodiments, an AL (e.g., a PAL) comprises a phenylalanine at a position corresponding to position 107 in SEQ ID NO: 1. In some embodiments, an AL (e.g., that is a PAL) comprises an aromatic, alkyl, and/or hydrophobic amino acid at a position corresponding to position 107 in SEQ ID NO: 1. In some embodiments, an AL (e.g., a PAL) comprises a leucine at a position corresponding to position 108 in SEQ ID NO: 1. In some embodiments, an AL (e.g., that is a PAL) comprises an aromatic, alkyl, and/or hydrophobic amino acid at a position corresponding to position 108 in SEQ ID NO: 1.

Without wishing to be bound by theory, the disclosure is directed, in part, to the idea that residues at positions corresponding to 107 and 108 of SEQ ID NO: 1 form a part of the active site of an AL, and that the presence of hydrophobic and/or packing (e.g., planar) amino acid side chains at these positions may preferentially stabilize phenylalanine (relative to tyrosine) in the active site, while the presence of polar side and/or packing amino acid side chains at these positions may preferentially stabilize tyrosine (relative to phenylalanine) in the active site. Such preferential stabilization may influence the specific activity of the AL for phenylalanine or tyrosine substrates. Accordingly, in some embodiments, an AL (e.g., a TAL) comprises aromatic, alkyl, and/or hydrophobic amino acids at positions corresponding to position 107 and/or 108 in SEQ ID NO: 1. In some embodiments, an AL comprises one or more amino acid substitutions replacing one or both of the naturally occurring amino acids at the positions corresponding to 107 and/or 108 in SEQ ID NO: 1 with aromatic, alkyl, and/or hydrophobic amino acids (e.g., that do not naturally occur at those sites), e.g., to preferentially process phenylalanine relative to tyrosine or to maintain preferential processing of phenylalanine relative to tyrosine.

In some embodiments, an AL, e.g., a PAL, is capable of assembling into a multimer (e.g., in a host cell). In some embodiments, a PAL is capable of assembling into a tetramer (e.g., in a host cell). The disclosure is further directed, in part, to a fusion polypeptide comprising a plurality of PALs, wherein the plurality of PALs is capable of multimerizing, e.g., with each other. In some embodiments, the fusion polypeptide comprising a plurality of PALs comprises 2, 3, 4, 5, 6, 7, or 8 PALs or functional fragments thereof. In some embodiments, the fusion polypeptide comprises a plurality of PALs wherein each PAL comprises the same amino acid sequence or is derived from either: naturally occurring PALs from the same organism, or the same naturally occurring PAL isoform. In some embodiments, the fusion polypeptide comprises a plurality of PALs comprising a first PAL and a second PAL, wherein the amino acid sequence of the first PAL is different from the amino acid sequence of the second PAL. In some embodiments, the fusion polypeptide comprises a plurality of PALs wherein each PAL is derived from a naturally occurring PAL from a different organism, or from different naturally occurring PAL isoforms from the same organism. As used in this context, derived includes making one or more alterations to the amino acid sequence of a naturally occurring PAL (e.g., a deletion (e.g., truncation), insertion, or substitution).

In some embodiments, an AL, e.g., a PAL, exhibits product inhibition, which refers to an inverse relationship between product (e.g., trans-cinnamic acid) concentration and the rate of the AL's production of product (e.g., trans-cinnamic acid) and/or consumption of substrate (e.g., L-phenylalanine). In some embodiments, an AL (e.g., a PAL) does not exhibit product inhibition or does not exhibit product inhibition with respect to PAL activity. In some embodiments, the amino acid sequence of a PAL comprises one or more modifications relative to a corresponding wildtype sequence that alter (e.g., decrease or eliminate) product inhibition. In some embodiments, an AL, e.g., a PAL, exhibits downstream product inhibition, which refers to an inverse relationship between a downstream product concentration and the rate of production of a product of the AL (e.g., trans-cinnamic acid) and/or consumption of substrate (e.g., L-phenylalanine). In some embodiments, a downstream product is any compound produced by an enzyme downstream of PAL in a metabolic pathway, e.g., the phenylpropanoid pathway. The downstream product may be produced by said metabolic pathway in a non-host cell (e.g., a cell comprising a naturally occurring PAL from which a PAL of the disclosure was derived), but the downstream product may be present in a host cell regardless of the presence of the metabolic pathway in the host cell. For example, a PAL may exhibit downstream product inhibition in a host cell from a downstream product of the phenylpropanoid pathway, because the downstream product is present in the host cell despite the absence of one or more components of the phenylpropanoid pathway.

In some embodiments, a downstream product includes, but is not limited to: p-coumarate, p-coumaroyl CoA, a stilbene, an isoflavonoid, a flavonol, a flavonol glycoside, caffeate, caffeic acid, methyl caffeic acid, ferulic acid, sinapic acid, a monolignol (e.g., p-coumaryl alcohol, coniferyl alcohol, or sinapyl alcohol), hesperetin dihydrochalcone 4′-O-glucoside (HDG), vanillin, vanillic acid, raspberry ketone, methyl cinnamate, naringenin and/or naringin, or derivatives thereof.

In some embodiments, a downstream product includes, but is not limited to: hydroxybenzalacetone, narirutin, phloretin, phloridzin, liquiritgenin, (2S)-flavanone, 2-hydroxy-flavanone, 7,4′-dihydroxyflavanone, 2-hydroxy-isoflavanone, formononetin, biochanin, 2′-hydroxy-formononetin, 4-coumaroyl-CoA, apigenin, chalconaringenin, daidzein, daidzin, malonyldaidzein (MGD), dihydrodaidzein, dihydrodaidzein-sulfate, O-desmethylangolensin, 6-OH—O-desmethylangolensin, tetrahydrodaidzein, equol, equol-7-glucuronide, equol-4′-sulfate, 5-hydroxy equol, hippuric acid, 4-hydroxybenzoic acid, 2,6-dimethoxy benzoic acid, fumaric acid, 4-ethylphenol, glutaric acid, 2-phenylpropionic acid, gallic acid, resorcinolsulfate, disometin, chrysoeriol, chrysoeriol-4′-glucuronide, chrysoeriol-7-glucuronide, coumestrol, eriodictyol, dihydroquercetin, genistein, genistin, malonylgenistin (MGG), glycitein, isorhamnetin, kaempferol, laricitrin, luteolin, luteolin-3′-glucuronide, luteolin-4′-glucuronide, morin, myricetin, tetramethylated myricetin, 3,5-dihydroxyphenylacetic acid, 3,4,5-trihydroxyphenylacetic acid, methylated myricetin, myricetin monoglucuronide, myricetin diglucuronide, dimethylated myricetin, pentahydroxy-flavanone, dihydromyricetin, 2R,3S,4S-flavan-3-ol, (+)-Afzelechin, (+)-catechin, (+)-galocatechin, proanthocyanidin, (−)-epiafzelechin, (−)-eoicatechin, (−)-epigallocatechin, taxifolin, dihydroquercetin, aromadendrin, dihydrokaempferol, dihydroquercetin, dihydroflavonol, quercetin, isoquercetin, rutin, peonidin, syringetin, tetrahydroxychalcone, trangeretin, chalcone, 6′-deoxychalcone, isoliquiritigenin, tetraketide, DHK, leuco-pelargonidin, pelargonidin, a pelargonidin-based anthocyanin, DHQ, leuco-cyanidin, cyanidin, a cyanidin-based anthocyanin, DHM, leuco-delphinidin, delphinidin, a delphidin-based anthocyanin, petunidin, malvidin, flavonol, flavone, flavanone, isoflavone, isoflavanone, and/or anthocyanin, or derivatives thereof.

In some embodiments, a downstream product includes, but is not limited to: cinnamate, methylcinnamate, cinnamoyl-CoA, cinnamaldehyde, styrene, pinocembrin chalcone, pinocembrin, chrysin, baicalein, curcumin, and/or bismethoxy curcumin, or derivatives thereof.

In some embodiments, a PAL does not exhibit downstream product inhibition. In some embodiments, the amino acid sequence of a PAL comprises one or more modifications relative to a corresponding wildtype sequence that alter (e.g., decrease or eliminate) downstream product inhibition.

In some embodiments, an AL, e.g., a PAL, capable of assembling into a multimer exhibits negative cooperativity with respect to binding and/or catalyzing conversion of L-phenylalanine. In some embodiments, an AL, e.g., a PAL, capable of assembling into a multimer does not exhibit negative cooperativity with respect to binding and/or catalyzing conversion of L-phenylalanine. In some embodiments, the amino acid sequence of a PAL comprises one or more modifications relative to a corresponding wildtype sequence that alter (e.g., decrease or eliminate) negative cooperativity. In some embodiments, a fusion polypeptide comprising a plurality of ALs, e.g., PALs, comprises PALs that do not exhibit negative cooperativity with respect to binding and/or catalyzing conversion of L-phenylalanine.

In some embodiments, an AL is a PAL from Anabaena variabilis (AvPAL) or a variant thereof (e.g., described herein). In some embodiments, a host cell comprises a PAL from Anabaena variabilis (AvPAL).

The Anabaena variabilis PAL is provided by SEQ ID NO: 1, which corresponds to the sequence provided by UniProtKB Accession No. Q3M5Z3 (expressed in strain t888841 described in the Examples):

MKTLSQAQSKTSSQQFSFTGNSSANVIIGNQKLTINDVARVARNGTLVSL
INNTDILQGIQASCDYINNAVESGEPIYGVTSGFGGMANVAISREQASEL
QTNLVWFLKTGAGNKLPLADVRAAMLLRANSHMRGASGIRLELIKRMEIF
LNAGVTPYVYEFGSIGASGDLVPLSYITGSLIGLDPSFKVDENGKEMDAP
TALRQLNLSPLTLLPKEGLAMMNGTSVMTGIAANCVYDTQILTAIAMGVH
ALDIQALNGTNQSFHPFIHNSKPHPGQLWAADQMISLLANSQLVRDELDG
KHDYRDHELIQDRYSLRCLPQYLGPIVDGISQIAKQIEIEINSVTDNPLI
DVDNQASYHGGNFLGQYVGMGMDHLRYYIGLLAKHLDVQIALLASPEFSN
GLPPSLLGNRERKVNMGLKGLQICGNSIMPLLTFYGNSIADREPTHAEQF
NQNINSQGYTSATLARRSVDIFQNYVAIALMFGVQAVDLRTYKKTGHYDA
RACLSPATERLYSAVRHVVGQKPTSDRPYIWNDNEQGLDEHIARISADIA
AGGVIVQAVQDILPCLH

A non-limiting example of a nucleotide sequence encoding SEQ ID NO: 1 is provided by SEQ ID NO: 2:

atgaaaacactgtcccaagcacagtcgaagacgagtagccagcagttttc
tttcaccggtaattctagcgctaacgtgattatcggcaaccagaaactaa
ctatcaacgacgtcgcccgcgttgctcggaacgggaccttagtaagcctg
actaacaacactgatatacttcaaggcattcaggcgtcctgtgattatat
caataacgcagttgagtctggtgaaccaatttacggtgtgacctcaggct
tcggcggtatggcgaacgttgctatcagccgtgaacaggcctccgaactg
caaaccaatttggtatggtttctgaaaactggtgctgggaacaaactccc
tttggcggacgtacgtgcagcaatgctgctgcgcgccaactcgcatatgc
gtggcgcgtccggtatccgtctggagctgatcaaacgtatggaaatcttc
ttgaacgctggtgttactccgtacgtttatgaatttggatctatcggcgc
ttctggagatctggtcccgctgagctacattacgggttccctgattggcc
ttgacccgagcttcaaggtggatttcaacggcaaagaaatggacgccccg
accgcgttacgccagttaaatctgtctcccctgacacttctgcctaaaga
aggtctagctatgatgaatgggacctcagtcatgactggcatcgcagcta
actgcgtatacgacacccagatcctgactgcgattgcaatgggtgttcac
gctctggatatccaggccctgaacggcaccaaccagtctttccacccgtt
tatccataactctaagccgcacccaggtcagctgtgggcggctgatcaga
tgatatcattgctggctaactcgcaactggtacgggacgagctggacggc
aaacatgattaccgcgaccacgagctgatccaggatcgttatagcctgcg
ttgccttccgcagtacctgggtccgattgtggacggtatctcacagatag
caaaacaaatcgaaattgaaattaactccgttactgataaccctctgatt
gacgtcgataaccaggcgtcgtaccacggcggaaatttcctgggtcagta
tgttggcatgggtatggaccaccttcgctactatatcggcctgctggcga
aacacctggatgtgcagattgcgctgctagctagtcccgaatttagcaac
ggactgccgccatctttattgggcaaccgtgaacgtaaggttaacatggg
tctgaaaggtttacaaatctgtggcaattccatcatgccgctgctgacgt
tctacggcaatagcatcgccgaccgctttccgacccatgcagagcaattc
aaccagaatatcaactctcagggctacacctccgcaacgctggcgcgacg
tagtgttgatatcttccaaaactacgttgcgattgccctgatgtttggcg
tccaggctgtagacctgaggacttataaaaagactggccattacgatgcg
cgtgcttgcctctctccggctaccgaacgcctgtattccgccgtgcgtca
cgtagttggtcagaaacctacttcagatcgcccatacatctggaacgata
acgagcagggtctggatgaacacatcgctcgcatctccgctgacattgcc
gctggcggagtaattgttcaagctgtacaggatatcctgccgtgcctgca
c

### Pal Variants for Increased Production of Trans-Cinnamic Acid

As described in Example 1, variant ALs that contain one or more amino acid substitutions relative to AvPAL (SEQ ID NO: 1) were identified in this disclosure that were capable of producing increased amounts of trans-cinnamic acid relative to AvPAL (SEQ ID NO: 1). Past efforts to improve AL activity have focused on improving in vivo AL activity via PEG-ylation of the AL (Hydery, T. and Coppenrath, V. A. (2019) “A Comprehensive Review of Pegvaliase, an Enzyme Substitution Therapy for the Treatment of Phenylketonuria”, Drug Target Insights). Aspects of the present disclosure relate to improvement of AL enzymatic activity to increase amounts of trans-cinnamic acid relative to a parent AL. The surprising and unexpected findings described in the present disclosure, including in Example 1, may lead to improved production of phenylpropanoid pathway products.

In some embodiments, an AL, e.g., a PAL, associated with the disclosure comprises at least 1, at least 2, at least 3, at least 4, at least 5, at least 6, at least 7, at least 8, at least 9, at least 10, at least 11, at least 12, at least 13, at least, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 60, at least 70, at least 80, at least 90, or at least 100 amino acid substitutions, deletions, insertions, or additions relative to SEQ ID NO: 1.

In some embodiments, a host cell that expresses a heterologous polynucleotide encoding an AL, e.g., a PAL, may increase conversion of L-phenylalanine to trans-cinnamic acid by 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) relative to a control. In some embodiments, the control is a host cell that expresses a heterologous polynucleotide encoding SEQ ID NO: 1.

In some embodiments, an AL, e.g., a PAL, comprises an amino acid sequence, or is encoded by a nucleic acid sequence, that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to any one of SEQ ID NOs: 1, 5-28, and 198-221, an amino acid or polynucleotide sequence of a PAL in Table 5, or a PAL otherwise described in this disclosure. In some embodiments, the amino acid sequence of an AL, e.g., a PAL, comprises or consists of any one of SEQ ID NOs: 1, 3, or 5-28 or a conservatively substituted version thereof.

In some embodiments, the sequence of an AL, e.g., a PAL, associated with the disclosure comprises one or more amino acid substitutions relative to SEQ ID NO: 1, wherein at least one of the amino acid substitutions is at a position corresponding to position 102, 104, 107, 108, 218, 219 and/or 222 in SEQ ID NO: 1.

In some embodiments, an AL, e.g., a PAL, comprises: a serine(S) at a position corresponding to position 102 in the sequence of SEQ ID NO: 1; a glutamic acid (E) at a position corresponding to position 102 in the sequence of SEQ ID NO: 1; a lysine (K) at a position corresponding to position 102 in the sequence of SEQ ID NO: 1; a histidine (H) at a position corresponding to position 102 in the sequence of SEQ ID NO: 1; a methionine (M) at a position corresponding to position 104 in the sequence of SEQ ID NO: 1; an alanine (A) at a position corresponding to position 104 in the sequence of SEQ ID NO: 1; an isoleucine (I) at a position corresponding to position 104 in the sequence of SEQ ID NO: 1; a valine (V) at a position corresponding to position 104 in the sequence of SEQ ID NO: 1; a histidine (H) at a position corresponding to position 107 in the sequence of SEQ ID NO: 1; a serine(S) at a position corresponding to position 107 in the sequence of SEQ ID NO: 1; a tyrosine (Y) at a position corresponding to position 107 in the sequence of SEQ ID NO: 1; a histidine (H) at a position corresponding to position 108 in the sequence of SEQ ID NO: 1; a threonine (T) at a position corresponding to position 108 in the sequence of SEQ ID NO: 1; a valine (V) at a position corresponding to position 108 in the sequence of SEQ ID NO: 1; a glutamine (Q) at a position corresponding to position 108 in the sequence of SEQ ID NO: 1; a methionine (M) at a position corresponding to position 108 in the sequence of SEQ ID NO: 1; an alanine (A) at a position corresponding to position 218 in the sequence of SEQ ID NO: 1; a serine(S) at a position corresponding to position 218 in the sequence of SEQ ID NO: 1; an isoleucine (I) at a position corresponding to position 219 in the sequence of SEQ ID NO: 1; a leucine (L) at a position corresponding to position 222 in the sequence of SEQ ID NO: 1; an asparagine (N) at a position corresponding to position 222 in the sequence of SEQ ID NO: 1; an isoleucine (I) at a position corresponding to position 222 in the sequence of SEQ ID NO: 1; a valine (V) at a position corresponding to position 222 in the sequence of SEQ ID NO: 1; a threonine (T) at a position corresponding to position 222 in the sequence of SEQ ID NO: 1; and/or any combination thereof.

In some embodiments, an AL, e.g., a PAL, comprises substitutions at: positions 102, 104, and 218 in the sequence of SEQ ID NO: 1; positions 104, 108, and 218 in the sequence of SEQ ID NO: 1; positions 102, 104, 108, 218, and 222 in the sequence of SEQ ID NO: 1; positions 102 and 222 in the sequence of SEQ ID NO: 1; positions 102, 104, and 219 in the sequence of SEQ ID NO: 1; positions 102, 108, and 222 in the sequence of SEQ ID NO: 1; positions 102, 108, 218, and 222 in the sequence of SEQ ID NO: 1; positions 102 and 218 in the sequence of SEQ ID NO: 1; positions 102, 104, 108, and 222 in the sequence of SEQ ID NO: 1; positions 102, 104, and 108 in the sequence of SEQ ID NO: 1; positions 102, 218, and 222 in the sequence of SEQ ID NO: 1; positions 102, 104, 219, and 222 in the sequence of SEQ ID NO: 1; positions 102 and 108 in the sequence of SEQ ID NO: 1; positions 104 and 222 in the sequence of SEQ ID NO: 1; positions 102, 108, and 218 in the sequence of SEQ ID NO: 1; or positions 104 and 108.

In some embodiments, an AL, e.g., a PAL, comprises the following amino acid substitutions relative to the sequence of SEQ ID NO: 1: T102H, L104M, and G218A; L104M, L108T, and G218A; T102E, L104M, L108T, G218A, and M222L; T102S and M222L; T102H, L104M, and L219I; T102H, L104M, L108T, G218A, and M222V; T102K and G218A; T102S, L108T, and M222L; T102S, L108T, G218S, and M222L; T102E, L108T, and M222I; T102E and G218S; T102K, L104I, L108T, and M222L; T102S, L104M, and L108M; T102K, G218A, and M222T; T102S, L104M, L219I, and M222L; T102H and L108T; L104M and M222V; T102H, L104M, G218A, and M222T; T102S, L108V, and G218A; L104A, L108T, and G218A; L104V and L108T; or T102K, L108V, and M222L.

In some embodiments, a host cell that expresses a heterologous polynucleotide encoding an AL, e.g., a PAL, may exhibit at least 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) more activity on L-phenylalanine relative to other amino acids. In some embodiments, a host cell that expresses a heterologous polynucleotide encoding an AL, e.g., a PAL, may exhibit at least 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) more activity on L-phenylalanine relative to other amino acids.

### Tyrosine Ammonia Lyases (TALs)

As described in Example 2, variant ALs were surprisingly identified in this disclosure that were active on L-tyrosine to produce p-coumaric acid. In some embodiments, an AL, including a variant AL associated with the disclosure, may be referred to as a “tyrosine ammonia lyase” or “TAL.” As used in this disclosure, a “tyrosine ammonia lyase” or “TAL” refers to an enzyme that catalyzes the conversion of L-tyrosine to ammonia and coumaric acid (FIG. 2). In some embodiments, a TAL is a L-tyrosine converting enzyme. Like other members of the aromatic amino acid lyase family of enzymes, naturally occurring TALs are characterized by the presence of a co-factor (4-methyldiene-imidazol-5-one (MIO)) in their active sites, formed in naturally occurring TALs by autocatalytic cyclization and dehydration of an internal tri-peptide segment (e.g., an Ala-Ser-Gly). TALs are found in a variety of microorganisms (e.g., cyanobacteria, bacteria (e.g., actinobacteria), and extremophiles), fungi (e.g., yeast), plants, and protists (e.g., algae), and are central to the phenylpropanoid pathway of plants, but do not naturally occur in mammalian animals such as humans. The phenylpropanoid pathway transforms aromatic amino acids produced from carbon sources in the shikimate pathway into a variety of different aromatic compounds; naturally occurring TAL produces coumaric acid from L-tyrosine, which can then be further processed by downstream enzymes such as, e.g., 4-coumarate-coenzyme A ligase, chalcone synthase, or flavonol synthase (FIG. 1). Naturally occurring TALs can have different substrate and/or product specificities; some predominantly deaminate L-tyrosine to ammonia and p-coumaric acid, whereas PALs from yeast and some monocot plants (e.g., maize) are known to convert L-phenylalanine and L-tyrosine to trans-cinnamic acid and p-coumaric acid, respectively. In a given plant species, multiple TAL-encoding genes may be found, increasing the number of naturally occurring TAL isoforms available for engineering. TAL enzymes occur as tetramers, with naturally occurring tetramers having molecular weights of about 64-478 kDa; heterotetramers of different naturally occurring TAL isoforms have been observed.

An AL of the disclosure that is a TAL can use L-tyrosine as a substrate. In some embodiments, an AL, e.g., a TAL, exhibits specificity for L-tyrosine compared to other amino acids (e.g., compared to L-phenylalanine or L-histidine). In some embodiments, a TAL produces ammonia and p-coumaric acid from L-tyrosine. In some embodiments, an AL, e.g., a TAL, predominantly consumes L-tyrosine relative to one or more other amino acids; e.g., may consume L-tyrosine at a rate at least 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, or 6-fold higher (e.g., 2-fold to 6-fold more) relative to one or more other amino acids (e.g., relative to L-phenylalanine or L-histidine). In some embodiments, an AL can convert L-phenylalanine into ammonia and trans-cinnamic acid. In some embodiments, an AL can convert L-histidine into ammonia and urocanic acid.

In some embodiments, an AL is selective for tyrosine (i.e., the AL is a TAL) when the phenylalanine residue at a position corresponding to position 107 in SEQ ID NO: 1 is substituted for a tyrosine and/or the leucine residue at a position corresponding to position 108 in SEQ ID NO: 1 is substituted for a histidine. Without wishing to be bound by any theory, substitutions at one or both of these residues may be involved in converting a PAL into a TAL. A phenylalanine residue at a position corresponding to position 107 in SEQ ID NO: 1 and/or a leucine residue at a position corresponding to position 108 of SEQ ID NO: 1 in a PAL may be more likely to effectively interact with the phenyl ring of L-phenylalanine, while a tyrosine residue at a position corresponding to position 107 in SEQ ID NO: 1 and/or a histidine residue at a position corresponding to position 108 of SEQ ID NO: 1 may be able to form hydrogen bonds with the hydroxyl functional group on L-tyrosine.

In some embodiments, an AL (e.g., a TAL) comprises an amino acid substitution at a position corresponding to position 107 and/or 108 in SEQ ID NO: 1. In some embodiments, an AL (e.g., a TAL) comprises a tyrosine at a position corresponding to position 107 in SEQ ID NO: 1. In some embodiments, an AL (e.g., that is a TAL) comprises an F107Y amino acid substitution relative to the sequence of SEQ ID NO: 1. In some embodiments, an AL (e.g., a TAL) comprises a histidine at a position corresponding to position 108 in SEQ ID NO: 1. In some embodiments, an AL (e.g., a TAL) comprises an L108H amino acid substitution relative to the sequence of SEQ ID NO: 1. In some embodiments, an AL (e.g., a TAL) comprises an amino acid substitution at a position corresponding to position 107 and/or 108 in SEQ ID NO: 1, wherein the substitution(s) replace one or both of the naturally occurring amino acids with polar and/or packing amino acids, e.g., to preferentially process tyrosine relative to phenylalanine.

In some embodiments, an AL, e.g., a TAL, is capable of assembling into a multimer (e.g., in a host cell). In some embodiments, a TAL is capable of assembling into a tetramer (e.g., in a host cell). The disclosure is further directed, in part, to a fusion polypeptide comprising a plurality of TALs, wherein the plurality of TALs is capable of multimerizing, e.g., with each other. In some embodiments, the fusion polypeptide comprising a plurality of TALs comprises 2, 3, 4, 5, 6, 7, or 8 TALs or functional fragments thereof. In some embodiments, the fusion polypeptide comprises a plurality of TALs wherein each TAL comprises the same amino acid sequence or is derived from either: naturally occurring TALs from the same organism, or the same naturally occurring TAL isoform. In some embodiments, the fusion polypeptide comprises a plurality of TALs comprising a first TAL and a second TAL, wherein the amino acid sequence of the first TAL is different from the amino acid sequence of the second TAL. In some embodiments, the fusion polypeptide comprises a plurality of TALs wherein each TAL is derived from a naturally occurring TAL from a different organism, or from different naturally occurring TAL isoforms from the same organism. As used in this context, derived includes making one or more alterations to the amino acid sequence of a naturally occurring TAL (e.g., a deletion (e.g., truncation), insertion, or substitution).

In some embodiments, an AL, e.g., a TAL, exhibits product inhibition, which refers to an inverse relationship between product (e.g., coumaric acid) concentration and the rate of the AL's production of product (e.g., coumaric acid) and/or consumption of substrate (e.g., L-tyrosine). In some embodiments, an AL, e.g., a TAL, does not exhibit product inhibition or does not exhibit product inhibition with respect to TAL activity. In some embodiments, the amino acid sequence of a TAL comprises one or more modifications relative to a corresponding wildtype sequence that alter (e.g., decrease or eliminate) product inhibition. In some embodiments, an AL, e.g., a TAL, exhibits downstream product inhibition, which refers to an inverse relationship between a downstream product concentration and the rate of production of a product of the AL (e.g., coumaric acid) and/or consumption of a substrate (e.g., L-tyrosine). In some embodiments, a downstream product is any compound produced by an enzyme downstream of TAL in a metabolic pathway, e.g., the phenylpropanoid pathway. The downstream product may be produced by said metabolic pathway in a non-host cell (e.g., a cell comprising a naturally occurring TAL from which a TAL of the disclosure was derived), but the downstream product may be present in a host cell regardless of the presence of the metabolic pathway in the host cell. For example, a TAL may exhibit downstream product inhibition in a host cell from a downstream product of the phenylpropanoid pathway, because the downstream product is present in the host cell despite the absence of one or more components of the phenylpropanoid pathway.

In some embodiments, a downstream product includes, but is not limited to: p-coumaroyl CoA, a stilbene, an isoflavonoid, a flavonol, a flavonol glycoside, caffeate, caffeic acid, methyl caffeic acid, ferulic acid, sinapic acid, or a monolignol (e.g., p-coumaryl alcohol, coniferyl alcohol, or sinapyl alcohol), p-coumaryl-CoA, dihydrocoumaroyl-CoA, phloretin, 3-hydroxyphloretin, hesperetin dihydrochalcone, or hesperetin dihydrochalcone 4′-O-glucoside (HDG), vanillin, vanillic acid, raspberry ketone, naringenin and/or naringin, or derivatives thereof.

In some embodiments, a downstream product includes, but is not limited to: hydroxybenzalacetone, narirutin, phloretin, phloridzin, liquiritgenin, (2S)-flavanone, 2-hydroxy-flavanone, 7,4′-dihydroxyflavanone, 2-hydroxy-isoflavanone, formononetin, biochanin, 2′-hydroxy-formononetin, 4-coumaroyl-CoA, apigenin, chalconaringenin, daidzein, daidzin, malonyldaidzein (MGD), dihydrodaidzein, dihydrodaidzein-sulfate, O-desmethylangolensin, 6-OH—O-desmethylangolensin, tetrahydrodaidzein, equol, equol-7-glucuronide, equol-4′-sulfate, 5-hydroxy equol, hippuric acid, 4-hydroxybenzoic acid, 2,6-dimethoxy benzoic acid, fumaric acid, 4-ethylphenol, glutaric acid, 2-phenylpropionic acid, gallic acid, resorcinolsulfate, disometin, chrysoeriol, chrysoeriol-4′-glucuronide, chrysoeriol-7-glucuronide, coumestrol, eriodictyol, dihydroquercetin, genistein, genistin, malonylgenistin (MGG), glycitein, isorhamnetin, kaempferol, laricitrin, luteolin, luteolin-3′-glucuronide, luteolin-4′-glucuronide, morin, myricetin, tetramethylated myricetin, 3,5-dihydroxyphenylacetic acid, 3,4,5-trihydroxyphenylacetic acid, methylated myricetin, myricetin monoglucuronide, myricetin diglucuronide, dimethylated myricetin, pentahydroxy-flavanone, dihydromyricetin, 2R,3S,4S-flavan-3-ol, (+)-Afzelechin, (+)-catechin, (+)-galocatechin, proanthocyanidin, (−)-epiafzelechin, (−)-eoicatechin, (−)-epigallocatechin, taxifolin, dihydroquercetin, aromadendrin, dihydrokaempferol, dihydroquercetin, dihydroflavonol, quercetin, isoquercetin, rutin, peonidin, syringetin, tetrahydroxychalcone, trangeretin, chalcone, 6′-deoxychalcone, isoliquiritigenin, tetraketide, DHK, leuco-pelargonidin, pelargonidin, a pelargonidin-based anthocyanin, DHQ, leuco-cyanidin, cyanidin, a cyanidin-based anthocyanin, DHM, leuco-delphinidin, delphinidin, a delphidin-based anthocyanin, petunidin, malvidin, flavonol, flavone, flavanone, isoflavone, isoflavanone, and/or anthocyanin, or derivatives thereof.

In some embodiments, a TAL does not exhibit downstream product inhibition. In some embodiments, a TAL does exhibit downstream product inhibition. In some embodiments, the amino acid sequence of a TAL comprises one or more modifications relative to a corresponding wildtype sequence that alter (e.g., decrease or eliminate) downstream product inhibition.

In some embodiments, an AL, e.g., a TAL, capable of assembling into a multimer exhibits negative cooperativity with respect to binding and/or catalyzing conversion of L-tyrosine. In some embodiments, an AL, e.g., a TAL, capable of assembling into a multimer does not exhibit negative cooperativity with respect to binding and/or catalyzing conversion of L-tyrosine. In some embodiments, the amino acid sequence of a TAL comprises one or more modifications relative to a corresponding wildtype sequence that alter (e.g., decrease or eliminate) negative cooperativity. In some embodiments, a fusion polypeptide comprising a plurality of ALs, e.g., TALs, comprises TALs that do not exhibit negative cooperativity with respect to binding and/or catalyzing conversion of L-tyrosine.

AL Variants with TAL Activity for Increased Production of Coumarate

As discussed above, Example 2 describes the surprising identification of variant ALs that were active on L-tyrosine to produce p-coumaric acid.

In some embodiments, an AL, e.g., a TAL, comprises at least 1, at least 2, at least 3, at least 4, at least 5, at least 6, at least 7, at least 8, at least 9, at least 10, at least 11, at least 12, at least 13, at least, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 60, at least 70, at least 80, at least 90, or at least 100 amino acid substitutions, deletions, insertions, or additions relative to SEQ ID NO: 1.

In some embodiments, a host cell that expresses a heterologous polynucleotide encoding an AL, e.g., a TAL, may increase conversion of L-tyrosine to p-coumaric acid by 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) relative to a control. In some embodiments, the control is a host cell that expresses a heterologous polynucleotide encoding SEQ ID NO: 1.

In some embodiments, an AL, e.g., a TAL, comprises an amino acid sequence, or is encoded by a nucleic acid sequence, that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to any one of SEQ ID NOs: 1-4, 29-197, or 222-388, an amino acid or polynucleotide sequence of a TAL in Table 5, or a TAL otherwise described in this disclosure. In some embodiments, the amino acid sequence of an AL, e.g., a TAL, comprises or consists of any one of SEQ ID NOs: 29-195 or a conservatively substituted version thereof.

In some embodiments, the sequence of an AL, e.g., a TAL, associated with the disclosure comprises one or more amino acid substitutions relative to SEQ ID NO: 1, wherein at least one of the amino acid substitutions is at a position corresponding to position 102, 104, 107, 108, 218, 219 and/or 222 in SEQ ID NO: 1.

In some embodiments, an AL, e.g., a TAL, comprises: a glutamic acid (E) at a position corresponding to position 102 in the sequence of SEQ ID NO: 1; a histidine (H) at a position corresponding to position 102 in the sequence of SEQ ID NO: 1; a lysine (K) at a position corresponding to position 102 in the sequence of SEQ ID NO: 1; a serine(S) at a position corresponding to position 102 in the sequence of SEQ ID NO: 1; an alanine (A) at a position corresponding to position 104 in the sequence of SEQ ID NO: 1; an isoleucine (I) at a position corresponding to position 104 in the sequence of SEQ ID NO: 1; a methionine (M) at a position corresponding to position 104 in the sequence of SEQ ID NO: 1; a valine (V) at a position corresponding to position 104 in the sequence of SEQ ID NO: 1; a histidine (H) at a position corresponding to position 107 in the sequence of SEQ ID NO: 1; a serine(S) at a position corresponding to position 107 in the sequence of SEQ ID NO: 1; a tyrosine (Y) at a position corresponding to position 107 in the sequence of SEQ ID NO: 1; a histidine (H) at a position corresponding to position 108 in the sequence of SEQ ID NO: 1; a methionine (M) at a position corresponding to position 108 in the sequence of SEQ ID NO: 1; a glutamine (Q) at a position corresponding to position 108 in the sequence of SEQ ID NO: 1; a threonine (T) at a position corresponding to position 108 in the sequence of SEQ ID NO: 1; a valine (V) at a position corresponding to position 108 in the sequence of SEQ ID NO: 1; an alanine (A) at a position corresponding to position 218 in the sequence of SEQ ID NO: 1; a serine(S) at a position corresponding to position 218 in the sequence of SEQ ID NO: 1; an isoleucine (I) at a position corresponding to position 219 in the sequence of SEQ ID NO: 1; an isoleucine (I) at a position corresponding to position 222 in the sequence of SEQ ID NO: 1; a leucine (L) at a position corresponding to position 222 in the sequence of SEQ ID NO: 1; an asparagine (N) at a position corresponding to position 222 in the sequence of SEQ ID NO: 1; a threonine (T) at a position corresponding to position 222 in the sequence of SEQ ID NO: 1; a valine (V) at a position corresponding to position 222 in the sequence of SEQ ID NO: 1.

In some embodiments, an AL, e.g., a TAL, comprises substitutions at: positions 104, 108, 219, and 222 in the sequence of SEQ ID NO: 1; positions 102, 108, 218, and 219 in the sequence of SEQ ID NO: 1 positions 102, 104, 108, 219, and 222 in the sequence of SEQ ID NO: 1; positions 102, 107, 108, 218, 219, and 222 in the sequence of SEQ ID NO: 1; positions 104, 108, 218, 219, and 222 in the sequence of SEQ ID NO: 1; positions 102, 104, 107, and 222 in the sequence of SEQ ID NO: 1; positions 102, 104, 107, 108, 219, and 222 in the sequence of SEQ ID NO: 1; positions 104, 218, and 222 in the sequence of SEQ ID NO: 1; positions 102, 108, 218, 219, and 222 in the sequence of SEQ ID NO: 1; positions 104, 108, and 218 in the sequence of SEQ ID NO: 1; positions 102, 107, 108, 219, and 222 in the sequence of SEQ ID NO: 1; positions 104, 107, 108, and 222 in the sequence of SEQ ID NO: 1; positions 102, 104, 108, 218, and 219 in the sequence of SEQ ID NO: 1; positions 102, 104, 107, 219, and 222 in the sequence of SEQ ID NO: 1; positions 102, 108, 218, and 222 in the sequence of SEQ ID NO: 1; positions 102, 108, and 222 in the sequence of SEQ ID NO: 1; positions 102, 104, 108, and 219 in the sequence of SEQ ID NO: 1; positions 102, 104, 107, 108, 218, 219, and 222 in the sequence of SEQ ID NO: 1; positions 102, 104, 107, 108, 218, and 219 in the sequence of SEQ ID NO: 1; positions 102, 107, 108, 219, and 222 in the sequence of SEQ ID NO: 1; positions 102, 104, 107, 108, 218, and 222 in the sequence of SEQ ID NO: 1; or positions 102, 104, 107, 108, and 219 in the sequence of SEQ ID NO: 1.

In some embodiments, an AL, e.g., a TAL, comprises the following amino acid substitutions relative to the sequence of SEQ ID NO: 1: L104A, L108Q, L219I, and M222N; T102S, L108Q, G218A, and L219I; T102H, L104M, L108M, L219I, and M222L; T102E, F107Y, L108M, G218S, L219I, and M222N; L104I, L108H, G218A, L219I, and M222V; T102E, L104M, F107Y, and M222I; T102E, L104V, F107Y, L108M, L219I, and M222T; T102S, L104I, G218S, L219I, and M222V; L104V, G218A, and M222L; T102K, L108H, G218A, L219I, and M222T; L104I, L108M, and G218S; T102H, F107Y, L108M, L219I, and M222V; L104V, F107H, L108Q, and M222L; T102K, L104A, L108Q, G218A, and L219I; T102S, L104A, F107S, L219I, and M222N; T102S, L108H, G218S, and M222V; T102K, L104A, L108H, L219I, and M222N; T102S, L108H, and M222N; T102H, L104M, L108M, and L219I; T102K, L104A, F107Y, L108V, G218A, L219I, and M222N; T102H, L108M, G218S, and M222L; T102E, L104M, F107Y, L108M, G218A, and L219I; T102E, L104V, F107H, and M222N; T102H, F107H, L108M, L219I, and M222T; T102H, L104V, F107S, L108Q, G218S, and M222T; T102E, L104M, F107S, L108M, G218A, and L219I; or T102E, L104V, F107Y, L108M, and L219I.

In some embodiments, an AL, e.g., a TAL, comprises the following amino acid substitutions relative to the sequence of SEQ ID NO: 1: T102E, L104V, F107Y, and L108H; T102E, F107Y, L108H, G218A, and M222I; T102S, F107Y, L108H, G218A, and M222T; T102E, L104M, F107Y, L108H, and G218A; L219I and M222T; F107Y, L108H, L219I, and M222T; L104A, L108Q, L219I, and M222N; T102S, L108Q, G218A, and L219I; T102H, L104M, L108M, and L219I; M222L; T102E, F107Y, L108M, and G218S; L219I and M222N; L104I, L108H, G218A, and L219I; M222V; T102E, L104M, F107Y, and M222I; T102E, F107Y, L108H, and M222I; T102E, F107Y, L108H, and G218A; T102S, F107Y, and L108H; T102E, F107Y, L108H, and M222T; or T102E, F107Y, L108H, and L219I.

In some embodiments, a host cell that expresses a heterologous polynucleotide encoding an AL, e.g., a TAL, may exhibit at least 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) more activity on L-tyrosine relative to other amino acids.

### Variants

Aspects of the disclosure relate to polynucleotides encoding any of the polypeptides, such as ALs (e.g., PALs and/or TALs), associated with the disclosure. Variants of polynucleotide or polypeptide sequences described in this application are also encompassed by the present disclosure. As used in this disclosure, a “variant” polynucleotide refers to a polynucleotide for which the nucleic acid sequence differs from the nucleic acid sequence of a reference polynucleotide by one or more changes in the nucleic acid sequence. As used in this disclosure, a “variant” polypeptide refers to a polypeptide for which the amino acid sequence differs from the amino acid sequence of a reference polypeptide by one or more changes in the amino acid sequence.

A variant polynucleotide or polypeptide can be constructed synthetically. Typically, the polynucleotide or polypeptide from which a variant is derived is a wild-type polynucleotide, a wild-type polypeptide, or a wild-type polynucleotide or polypeptide domain. However, the variants usable in the present disclosure may also be derived from homologs, orthologs, or paralogs of a wild-type polynucleotide, a wild-type polypeptide, or a wild-type polynucleotide or polypeptide domain, or from synthetic polynucleotides or polypeptides. The changes in the nucleic acid and/or amino acid sequences may include substitutions, insertions, deletions, N-terminal truncations, C-terminal truncations, N-terminal additions, C-terminal additions, or any combination of these changes, which may occur at one or multiple positions.

A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity” refers to the relatedness of the sequences of two polypeptides or polynucleotides when the sequences are aligned, and the term “percent identity” refers to the percentage of residues (amino acids or nucleotides) that are identical when two or more polypeptide or polynucleotide sequences are aligned. In some embodiments, sequence identity and/or percent identity is determined across the entire length of a sequence, while in other embodiments, sequence identity and/or percent identity is determined over a region of a sequence.

Percent identity of polypeptide or polynucleotide sequences can be calculated by any of the methods known to one of ordinary skill in the art. For example, percent identity can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3. Where gaps exist between two sequences, Gapped BLAST® can be utilized, for example, as described in Altschul et al., Nucleic Acids Res. 25 (17): 3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

A second example of a local alignment technique is based on the Smith-Waterman algorithm (Smith, T. F. & Waterman, M. S. (1981) “Identification of common molecular subsequences.” J. Mol. Biol. 147:195-197). An example of a global alignment technique is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming. A further example of a global alignment technique is the Fast Optimal Global Sequence Alignment Algorithm (FOGSAA).

In some embodiments, the identity of two polypeptide sequences is determined by aligning the two amino acid sequences of the polypeptides, calculating the number of identical amino acids, and dividing by the length of one of the polypeptide sequences. In some embodiments, the identity of two polynucleotide sequences is determined by aligning the two nucleotide sequences of the polynucleotides, calculating the number of identical nucleotides and dividing by the length of one of the polynucleotide sequences.

For multiple sequence alignments, computer programs including Clustal Omega (Sievers et al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453).

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA).

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539).

Functional variants of ALs, PALs, TALs, and any other proteins disclosed in this application are also encompassed by the present disclosure. As used in this disclosure, a functional variant of an AL, PAL, or a TAL refers to an AL, PAL, or TAL that has a different sequence than the sequence of a reference AL, PAL, or TAL but that maintains, partially or fully, at least one activity of the reference AL, PAL, or TAL. In some embodiments, a functional variant of an AL, PAL, or TAL enhances one or more activities of a reference AL, PAL, or TAL. For example, a functional variant may bind one or more of the same substrates (e.g., phenylalanine, tyrosine, or precursors thereof) or produce one or more of the same products (e.g., trans-cinnamic acid or p-coumaric acid).

Variant sequences, including functional variants, may be homologous sequences. Homologous sequences include but are not limited to paralogous sequences, orthologous sequences, or sequences arising from convergent evolution. Paralogous sequences arise from duplication of a gene within a genome of a species, while orthologous sequences diverge after a speciation event. Two different species may have evolved independently but may each comprise a sequence that shares a certain percent identity with a sequence from the other species as a result of convergent evolution. As used in this disclosure, a functional homolog of a reference AL, PAL, or TAL maintains, partially or fully, at least one activity of the reference AL, PAL, or TAL. In some embodiments, a functional homolog of an AL, PAL, or TAL enhances one or more activities of a reference AL, PAL, or TAL. For example, a functional homolog may bind one or more of the same substrates (e.g., phenylalanine, tyrosine, or precursors thereof) or produce one or more of the same products (e.g., trans-cinnamic acid or p-coumaric acid).

Functional variants may be variants of naturally occurring sequences. Functional variants can also be created via site-directed mutagenesis of the coding sequence for a polypeptide, or by combining domains from the coding sequences for different naturally-occurring polypeptides (“domain swapping”). Techniques for modifying genes encoding functional variants described in this disclosure are known in the art and include, inter alia, directed evolution techniques, site-directed mutagenesis techniques and random mutagenesis techniques, and can be useful, for example, to increase specific activity of a polypeptide, alter substrate specificity, alter expression levels, alter subcellular location, or modify polypeptide: polypeptide interactions in a desired manner.

Variants and homologs can be identified by analysis of polynucleotide and polypeptide sequence alignments. For example, performing a query on a database of polynucleotide or polypeptide sequences can identify variants and homologs of polynucleotide sequences encoding derivative polypeptides and the like.

Hybridization can also be used to identify functional variants or functional homologs and/or as a measure of homology between two polynucleotide sequences. A polynucleotide sequence encoding any of the polypeptides disclosed in this application, or a portion thereof, can be used as a hybridization probe according to standard hybridization techniques. The hybridization of a probe to DNA or RNA from a test source (e.g., a mammalian cell) is an indication of the presence of the relevant DNA or RNA in the test source. Hybridization conditions are known to those skilled in the art and can be found in, e.g., Current Protocols in Molecular Biology, John Wiley & Sons, N.Y., 6.3.1-6.3.6, 1991. In some embodiments, moderate hybridization conditions include hybridization in 2× sodium chloride/sodium citrate (SSC) at 30° C. followed by a wash in 1×SSC, 0.1% SDS at 50° C. In some embodiments, highly stringent conditions include hybridization in 6× sodium chloride/sodium citrate (SSC) at 45° C. followed by a wash in 0.2×SSC, 0.1% SDS at 65° C.

Sequence analysis to identify functional variants or functional homologs can also involve BLAST, Reciprocal BLAST, or PSI-BLAST analysis of non-redundant databases using a relevant amino acid sequence as the reference sequence. An amino acid sequence is, in some instances, deduced from a polynucleotide sequence. In some embodiments, polypeptides that have greater than 40% sequence identity may be identified as candidates for further evaluation for suitability for use according to the disclosure. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have, e.g., conserved functional domains.

In some embodiments, a polypeptide variant (e.g., AL, PAL, or TAL variant or variant of any other polypeptide associated with the disclosure) comprises a domain that shares a secondary structure (e.g., alpha helix, beta sheet) with a reference polypeptide (e.g., a reference AL, PAL, or TAL, or any other polypeptide associated with the disclosure). In some embodiments, a polypeptide variant (e.g., AL, PAL, or TAL variant or variant of any other polypeptide associated with the disclosure) shares a tertiary structure with a reference polypeptide (e.g., a reference AL, PAL, or TAL, or any other polypeptide associated with the disclosure). In some embodiments, a reference polypeptide is an AL, e.g., a PAL, comprising the sequence of SEQ ID NO: 1. As a non-limiting example, a variant polypeptide may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets, or have the same tertiary structure as a reference polypeptide. For example, a loop may be located between a beta sheet and an alpha helix, between two alpha helices, or between two beta sheets. Homology modeling may be used to compare two or more tertiary structures.

Mutations can be made in a nucleotide sequence by any method known to one of ordinary skill in the art. For example, mutations can be made by gene editing tools, PCR, site-directed mutagenesis (e.g., according to Kunkel, Proc. Nat. Acad. Sci. U.S.A. 82: 488-492, 1985), chemical synthesis of a gene or polypeptide, or by insertions, such as insertion of a tag (e.g., a HIS tag or a GFP tag). Mutations can include, for example, substitutions, deletions, additions, insertions, fusions, and translocations, generated by any method known in the art.

In some embodiments, methods for producing variants include circular permutation (Yu and Lutz, Trends Biotechnol. 2011 January; 29(1):18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) compared to the linear sequence of the polypeptide before it was circularized and severed as determined by linear sequence alignment methods (e.g., Clustal Omega or BLAST). Topological analysis of the two polypeptides, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce a polypeptide with different functional characteristics (e.g., increased or decreased enzymatic activity, different substrate specificity, or different product specificity). See, e.g., Yu and Lutz, Trends Biotechnol. 2011 January; 29(1):18-25.

It should be appreciated that in a polypeptide that has undergone circular permutation, the linear amino acid sequence of the polypeptide would differ from a reference polypeptide that has not undergone circular permutation. However, one of ordinary skill in the art would be able to determine which residues in the polypeptide that has undergone circular permutation correspond to residues in the reference polypeptide that has not undergone circular permutation by, for example, aligning the sequences and detecting conserved motifs, and/or by comparing the structures or predicted structures of the polypeptides, e.g., by homology modeling.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr. 1; 21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

Functional variants or functional homologs may be identified using any method known in the art. For example, the algorithm of Karlin and Altschul Proc. Natl. Acad. Sci. USA 87:2264-68, 1990 described above may be used to identify homologous proteins.

Putative functional variants or functional homologs may also be identified by searching for polypeptides with functionally annotated domains. Databases including Pfam (Sonnhammer et al., Proteins. 1997 July; 28(3):405-20) may be used to identify polypeptides with a particular domain.

Homology modeling may also be used to identify amino acid residues that are amenable to mutation without affecting function. A non-limiting example of such a method may include use of position-specific scoring matrix (PSSM) and an energy minimization protocol. See, e.g., Stormo et al., Nucleic Acids Res. 1982 May 11; 10(9):2997-3011.

PSSM may be paired with calculation of a Rosetta energy function, which determines the difference between the wild-type and a mutant, such as a point mutant. Without being bound by a particular theory, potentially stabilizing mutations can be desirable for protein engineering (e.g., production of functional homologs). In some embodiments, a potentially stabilizing mutation has a ΔΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul. 21; 63 (2): 337-346. doi: 10.1016/j.molcel.2016.06.012.

In some embodiments, a polynucleotide sequence encoding an AL, e.g., a PAL and/or TAL, or a polynucleotide sequence encoding any other polypeptide associated with the disclosure comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 nucleotide positions corresponding to a reference sequence. In some embodiments, the polynucleotide sequence encoding the AL, e.g., PAL and/or TAL, or the polynucleotide sequence encoding any other polypeptide associated with the disclosure comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of a coding sequence relative to a reference coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence relative to the amino acid sequence of a reference polypeptide.

In some embodiments, the one or more mutations in a polynucleotide sequence encoding an AL, e.g., a PAL and/or TAL, or encoding any other polypeptide associated with the disclosure, alter the amino acid sequence of the polypeptide relative to the amino acid sequence of a reference polypeptide. In some embodiments, the one or more mutations alter the amino acid sequence of the recombinant polypeptide relative to the amino acid sequence of a reference polypeptide and alter (enhance or reduce) an activity of the polypeptide relative to the reference polypeptide.

Assays for determining and quantifying enzyme and/or enzyme variant activity are described herein and are known in the art. By way of example, enzyme and/or enzyme variant activity can be determined by incubating a purified enzyme or enzyme variant or extracts from host cells or a complete recombinant host organism that has produced the enzyme or enzyme variant with an appropriate substrate under appropriate conditions and carrying out an analysis of the reaction products (e.g., by gas chromatography (GC) or liquid chromatography (LC) analysis). Further details on enzyme and/or enzyme variant activity assays and analysis of the reaction products are provided in the Examples. These assays include producing enzyme variants in recombinant host cells.

The activity, including specific activity, of any of the enzymes described in this application may be measured using methods known in the art. As a non-limiting example, an enzyme's activity may be determined by measuring its substrate specificity, product(s) produced, the concentration of product(s) produced, or any combination thereof.

As used in this disclosure, the term “activity” means the ability of an enzyme to react with a substrate to provide a target product. The activity of an enzyme can be determined in an activity test via measuring the increase of one or more target products, the decrease of one or more substrates (or starting materials) or via measuring a combination of these parameters as a function of time. As used in this application, “specific activity” of an enzyme refers to the amount (e.g., concentration) of a particular product produced for a given amount (e.g., concentration) of the enzyme per unit time.

A “biological activity” as used in this disclosure, refers to any activity a polypeptide may exhibit, including without limitation: enzymatic activity; binding activity to another compound (e.g., binding to another polypeptide, in particular binding to a receptor, or binding to a nucleic acid); inhibitory activity (e.g., enzyme inhibitory activity); activating activity (e.g., enzyme-activating activity); or toxic effects. In some embodiments, a functional variant polypeptide exhibits the relevant activity to a degree of at least 10% of the activity of the parent or reference polypeptide.

In some embodiments, a functional variant of an enzyme associated with the present disclosure produces a better yield than a reference or parent enzyme (e.g., a wild-type enzyme or a reference enzyme variant). As used in this disclosure, the term “yield” refers to the gram of recoverable product per gram of feedstock (which can be calculated as a percent molar conversion rate).

In some embodiments, a functional variant of an enzyme associated with the present disclosure exhibits modified (e.g., increased) productivity relative to a reference or parent enzyme (e.g., a wild-type enzyme or a reference enzyme variant).

As used in this disclosure, “productivity” of a variant AL, e.g., PAL and/or TAL, refers to the fold increase in production of a desired product by the variant AL relative to the production of the desired product by a reference or parent enzyme (e.g., a wild-type enzyme or a reference enzyme variant). For example, when the desired product is trans-cinnamic acid or p-coumaric acid, then productivity of a variant AL refers to the fold increase in production of trans-cinnamic acid or p-coumaric acid by the variant AL relative to the production of trans-cinnamic acid or p-coumaric acid by a reference or parent enzyme (e.g., a wild-type enzyme or a reference enzyme variant).

In some embodiments, a functional variant of an enzyme associated with the present disclosure exhibits a modified (e.g., increased) target productivity relative to a reference or parent enzyme. The term “target productivity” refers to the amount of recoverable target product in grams per liter of fermentation capacity per hour of bioconversion time (i.e., time after the substrate was added).

In some embodiments, a functional variant of an enzyme associated with the present disclosure exhibits a modified target yield factor relative to a reference or parent enzyme. The term “target yield factor” refers to the ratio between the product concentration obtained and the concentration of the variant/derivative (for example, purified enzyme or an extract from a recombinant host cell expressing the desired enzyme) in culture medium.

In some embodiments, a functional variant of an enzyme associated with the present disclosure exhibits a modified (e.g., increased) fold in enzymatic activity relative to a reference or parent enzyme (e.g., SEQ ID NO: 1). In some embodiments, the increase in activity is by at least a factor of: 2, 3, 4, 6, 8, 10, 12, 14, 16, 18, 20, 25, 30, 35, 40, 45, 50, 55, 60, 65, 70, 75, 80, 85, 90, 95, 100 or more than 100.

In some embodiments, a functional variant of an enzyme associated with the present disclosure exhibits a modified (e.g., increased) target productivity relative to a reference or parent enzyme. The term “target productivity” refers to the amount of recoverable target product in grams per liter of fermentation capacity per hour of bioconversion time (i.e., time after the substrate was added).

Mutations in a polypeptide coding sequence may result in conservative amino acid substitutions. As used in this application, a “conservative amino acid substitution” or “conservatively substituted amino acid” refers to an amino acid substitution that does not alter the relative charge or size characteristics or functional activity of the protein in which the amino acid substitution is made.

Accordingly, as used in this disclosure, the term “conservative amino acid substitution” means an exchange of an amino acid by another amino acid listed within the same group of the six standard amino acid groups shown below.
(1) hydrophobic (non-polar): Met, Ala, Val, Leu, Ile, Gly, Pro, Trp, Phe;
(2) neutral hydrophilic: Cys, Ser, Thr; Asn, Gln, Tyr;
(3) acidic: Asp, Glu;
(4) basic: His, Lys, Arg;
(5) residues that influence chain orientation: Gly, Pro;
(6) aromatic: Trp, Tyr, Phe.

For example, the exchange of Asp by Glu retains one negative charge in the modified polypeptide. In addition, glycine and proline may be substituted for one another based on their ability to disrupt alpha-helices. Some preferred conservative substitutions within the above six groups are exchanges within the following sub-groups: (i) Ala, Val, Leu and Ile; (ii) Ser and Thr; (ii) Asn and Gln; (iv) Lys and Arg; and (v) Tyr and Phe. Given the known genetic code, and recombinant and synthetic DNA techniques, the skilled scientist readily can construct polynucleotide sequences encoding conservatively substituted amino acid variants.

As used herein, “non-conservative amino acid substitutions” or “non-conservative amino acid exchanges” are defined as exchanges of an amino acid by another amino acid listed in a different group of the six standard amino acid groups (1) to (6) as shown above. In some embodiments, variants of enzymes associated with the present disclosure are prepared using non-conservative substitutions that alter the biological function of the variants.

For ease of reference, the one-letter amino acid symbols recommended by the IUPAC-IUB Biochemical Nomenclature Commission are indicated as follows. The three letter codes are also provided for reference purposes.

TABLE 1
Amino Acid Symbols

| One Letter Code | Three Letter Code | Amino acid name |
|---|---|---|
| A | Ala | Alanine |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

Amino acid alterations such as amino acid substitutions may be introduced using known protocols of recombinant gene technology including PCR, gene cloning, site-directed mutagenesis of cDNA, transfection of host cells, and in-vitro transcription which may be used to introduce such changes to a sequence resulting in a variant/derivative enzyme. Variants containing amino acid alterations can be screened for functional activity.

In some instances, an amino acid is characterized by its R group (see, e.g., Table 2). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Functionally equivalent variants of polypeptides may include conservative amino acid substitutions. Non-limiting examples of conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Additional non-limiting examples of conservative amino acid substitutions are provided in Table 2.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 2
Non-limiting examples of conservative amino acid substitutions

| | | Conservative Amino |
|---|---|---|
| Original Residue | R Group Type | Acid Substitutions |
| Ala (A) | nonpolar aliphatic R group | Cys, Gly, Ser |
| Arg (R) | positively charged R group | His, Lys |
| Asn (N) | polar uncharged R group | Asp, Gln, Glu |
| Asp (D) | negatively charged R group | Asn, Gln, Glu |
| Cys (C) | polar uncharged R group | Ala, Ser |
| Gln (Q) | polar uncharged R group | Asn, Asp, Glu |
| Glu (E) | negatively charged R group | Asn, Asp, Gln |
| Gly (G) | nonpolar aliphatic R group | Ala, Ser |
| His (H) | positively charged R group | Arg, Tyr, Trp |
| Ile (I) | nonpolar aliphatic R group | Len, Met, Val |
| Leu (L) | nonpolar aliphatic R group | Ile, Met, Val |
| Lys (K) | positively charged R group | Arg, His |
| Met (M) | nonpolar aliphatic R group | Ile, Leu, Phe, Val |
| Pro (P) | polar uncharged R group | |
| Phe (F) | nonpolar aromatic R group | Met, Trp, Tyr |
| Ser (S) | polar uncharged R group | Ala, Gly, Thr |
| Thr (T) | polar uncharged R group | Ala, Asn, Ser |
| Trp (W) | nonpolar aromatic R group | His, Phe, Tyr, Met |
| Tyr (Y) | nonpolar aromatic R group | His, Phe, Trp |
| Val (V) | nonpolar aliphatic R group | Ile, Leu, Met, Thr |

In some embodiments of the disclosure, an amino acid at a particular position in a protein may be replaced by an amino acid that has a different molecular weight. For example, in some embodiments, an amino acid at a particular position in a protein may be replaced by a “larger” amino acid, which refers to an amino acid that has a larger molecular weight. In other embodiments, an amino acid at a particular position in a protein may be replaced by a “smaller” amino acid, which refers to an amino acid that has a smaller molecular weight. The amino acids, ranked from smallest to largest based on molecular weight are: G, A, S, P, V, T, C, I, L, N, D, E, K, Q, M, H, F, R, Y, and W.

Amino acid substitutions in the amino acid sequence of a polypeptide to produce a polypeptide variant having a desired property and/or activity can be made by alteration of the coding sequence of the polypeptide. Similarly, conservative amino acid substitutions in the amino acid sequence of a polypeptide to produce functionally equivalent variants of the polypeptide typically are made by alteration of the coding sequence of the polypeptide (e.g., PAL or TAL, or any other polypeptide associated with the disclosure).

### Polynucleotides Encoding ALs

Aspects of the present disclosure relate to recombinant enzymes, functional modifications and variants thereof, polynucleotides encoding said enzymes, as well as uses relating to any thereof. For example, the enzymes and cells described in this application may be used to promote L-phenylalanine and/or L-tyrosine processing, e.g., by converting L-phenylalanine to trans-cinnamic acid and/or by converting L-tyrosine to p-coumaric acid. The methods may comprise using a host cell comprising one or more enzymes disclosed in this application, a cell lysate, isolated enzymes, or any combination thereof. Methods comprising recombinant expression of polynucleotides encoding an enzyme disclosed in this application in a host cell are encompassed by the present disclosure. In vitro methods comprising reacting one or more ALs, e.g., PALs and/or TALs, in a reaction mixture disclosed in this application are also encompassed by the present disclosure.

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system; or a polynucleotide whose expression or regulation has been manipulated within a biological system. A heterologous polynucleotide that is introduced into or expressed in a host cell may be a polynucleotide that comes from a different organism or species from the host cell, or may be a synthetic polynucleotide, or may be a polynucleotide that is also endogenously expressed in the same organism or species as the host cell. For example, a polynucleotide that is endogenously expressed in a host cell may be considered heterologous when it is: situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez et al., Nat Methods. 2016 July; 13(7): 563-567. A heterologous polynucleotide may comprise a wild-type sequence or a mutant sequence as compared with a reference polynucleotide sequence.

A polynucleotide encoding any of the polypeptides, such as PALs or TALs, or any other polypeptides associated with the disclosure, may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector). The vector may be a cloning vector, such as a plasmid, fosmid, phagemid, virus genome or artificial chromosome.

As used in this application, the terms “expression vector” or “expression construct” refer to a nucleic acid construct, generated recombinantly or synthetically, with a series of specified nucleic acid elements that permit transcription of a particular polynucleotide in a host cell, such as a yeast cell or bacterial cell. In some embodiments, a polynucleotide associated with the disclosure is inserted into an expression vector or expression construct such that it is operably joined to regulatory sequences and, in some embodiments, expressed as an RNA transcript. In some embodiments, the expression vector or expression construct contains one or more markers, such as a selectable marker, to identify cells transformed or transfected with the expression vector or expression construct. A polynucleotide encoding a polypeptide associated with the disclosure is “operably joined” or “operably linked” to a regulatory sequence when the polynucleotide and the regulatory sequence are covalently linked and the expression or transcription of the polynucleotide is under the influence or control of the regulatory sequence.

In some embodiments, a polynucleotide encoding any of the polypeptides described in this application is under the control of regulatory sequences (e.g., enhancer sequences). In some embodiments, a polynucleotide (e.g., a polynucleotide comprising a gene) is expressed under the control of a promoter. In some embodiments, the promoter is a native promoter, corresponding to the promoter of the gene in its endogenous context. In other embodiments, the promoter is not the native promoter of the gene, e.g., the promoter is different from the promoter of the gene in its endogenous context.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TPI1 GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls1con, T3, T7, SP6, and PL. Non-limiting examples of bacterial promoters include Pbad, PmgrB, Ptrc2, Plac/ara, Ptac, and Pm.

In some embodiments, the promoter is an inducible promoter. As used in this application, an “inducible promoter” is a promoter controlled by the presence or absence of a molecule. Non-limiting examples of inducible promoters include chemically-regulated promoters and physically-regulated promoters. For chemically-regulated promoters, the transcriptional activity can be regulated by one or more compounds, such as alcohol, an antibiotic such as tetracycline, a carbon source such as galactose, a steroid, a metal, or other compounds. For physically-regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1, TPI1, HXT3, HXT7, ACT1, ADH1, ADH2, ENO2, and SOD1.

Other inducible promoters or constitutive promoters known to one of ordinary skill in the art are also contemplated.

In some embodiments, introduction of a polynucleotide, such as a polynucleotide encoding a polypeptide associated with the disclosure, into a host cell results in genomic integration of the polynucleotide. In some embodiments, a host cell comprises at least 1 copy, at least 2 copies, at least 3 copies, at least 4 copies, at least 5 copies, at least 6 copies, at least 7 copies, at least 8 copies, at least 9 copies, at least 10 copies, at least 11 copies, at least 12 copies, at least 13 copies, at least 14 copies, at least 15 copies, at least 16 copies, at least 17 copies, at least 18 copies, at least 19 copies, at least 20 copies, at least 21 copies, at least 22 copies, at least 23 copies, at least 24 copies, at least 25 copies, at least 26 copies, at least 27 copies, at least 28 copies, at least 29 copies, at least 30 copies, at least 31 copies, at least 32 copies, at least 33 copies, at least 34 copies, at least 35 copies, at least 36 copies, at least 37 copies, at least 38 copies, at least 39 copies, at least 40 copies, at least 41 copies, at least 42 copies, at least 43 copies, at least 44 copies, at least 45 copies, at least 46 copies, at least 47 copies, at least 48 copies, at least 49 copies, at least 50 copies, at least 60 copies, at least 70 copies, at least 80 copies, at least 90 copies, at least 100 copies, or more, including any values in between, of a polynucleotide sequence, such as a polynucleotide sequence encoding any of the polypeptides described in this application, in its genome. Said copies may be inserted into the same locus or into different loci of a recombinant host cell of the disclosure.

In some embodiments, the sequence of a polynucleotide (e.g., a polynucleotide comprising a gene) is codon-optimized. Codon optimization may increase expression of a gene by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not codon-optimized.

In some embodiments, a polynucleotide encoding a PAL comprises a sequence that is at least 50% (e.g., at least 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more than 99%, including all values in between) identical to any one of SEQ ID NOs: 40-76 or 93-108. In certain embodiments, a polynucleotide encoding a PAL comprises any one of SEQ ID NOs: 2 or 198-221. In certain embodiments a polynucleotide encoding a PAL consists of or consists essentially of any one of SEQ ID NOs: 2 or 198-221.

In some embodiments, a polynucleotide encoding a TAL comprises a sequence that is at least 50% (e.g., at least 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more than 99%, including all values in between) identical to any one of SEQ ID NOs: 40-76 or 93-108. In certain embodiments, a polynucleotide encoding a TAL comprises any one of SEQ ID NOs: 2 or 222-388. In certain embodiments a polynucleotide encoding a TAL consists of or consists essentially of any one of SEQ ID NOs: SEQ ID NOs: 2 or 222-388.

### Host Cells

Any of the polynucleotides or polypeptides of the disclosure may be expressed in a host cell. As used in this application, the term “host cell” refers to a cell that can be used to express a polynucleotide, such as a polynucleotide that encodes a polypeptide used in production of trans-cinnamic acid and/or p-coumaric acid and precursors thereof.

Any suitable host cell may be used to express any of the recombinant polypeptides, including ALs, PALs, or TALs, and other polypeptides disclosed in this application, including eukaryotic cells or prokaryotic cells. Suitable host cells include, but are not limited to, fungal cells (e.g., yeast cells), bacterial cells (e.g., E. coli cells), algal cells, plant cells, insect cells, and animal cells, including mammalian cells.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Campylobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host strain is an industrial strain. Numerous bacterial industrial strains are known and suitable for the methods and compositions described in this application.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans, B. amyloliquefaciens). In particular embodiments, the host cell will be an industrial Bacillus strain including but not limited to B. subtilis, B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell will be an industrial Zymomonas species (e.g., Z. mobilis, Z. lipolytica), and the like.

The present disclosure is also suitable for use with a variety of animal cell types, including mammalian cells, for example, human (including 293, HeLa, WI38, PER.C6 and Bowes melanoma cells), mouse (including 3T3, NS0, NS1, Sp2/0), hamster (CHO, BHK), monkey (COS, FRhL, Vero), and hybridoma cell lines.

In various embodiments, cell types or strains that may be used in the practice of the disclosure including both prokaryotic and eukaryotic cell or strains, and are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL). The present disclosure is also suitable for use with a variety of plant cell types.

The term “cell,” as used in this application, may refer to a single cell or a population of cells, such as a population of cells belonging to the same cell line or strain. Use of the singular term “cell” should not be construed to refer explicitly to a single cell rather than a population of cells. The host cell may comprise genetic modifications relative to a wild-type counterpart.

A vector or polynucleotide encoding any one or more of the recombinant polypeptides (e.g., AL, PAL, or TAL) described in this application may be introduced into a suitable host cell using any method known in the art. Host cells may be cultured under any conditions suitable as would be understood by one of ordinary skill in the art. For example, any media, temperature, and incubation conditions known in the art may be used. For host cells carrying an inducible vector, cells may be cultured with an appropriate inducible agent to promote expression.

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermenter is used to culture the cell. Thus, in some embodiments, the cells are used in fermentation. As used in this application, the terms “bioreactor” and “fermenter” are interchangeably used and refer to an enclosure, or partial enclosure, in which a biological, biochemical and/or chemical reaction takes place, involving a living organism or part of a living organism. Any type of bioreactor or fermenter known in the art may be compatible with aspects of the disclosure.

In some embodiments, a bioreactor comprises a cell (e.g., a bacterial cell) or a cell culture (e.g., a bacterial cell culture), such as a cell or cell culture described in this application. In some embodiments, a bioreactor comprises a spore and/or a dormant cell type of an isolated microbe (e.g., a dormant cell in a dry state).

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product may display some differences from the substrate in terms of solubility, toxicity, cellular accumulation and secretion and in some embodiments can have different fermentation kinetics.

Any suitable host cell may be used to produce any of the recombinant polypeptides (e.g., AL, e.g., PAL and/or TAL) disclosed in this application, including eukaryotic cells or prokaryotic cells.

The disclosure is directed, in part, to host cells comprising polynucleotides encoding a plurality of enzymes with activities that together promote production of an aromatic compound or improve an aromatic compound manufacturing mixture. For example, the disclosure provides a host cell comprising a polynucleotide encoding an AL (e.g., a PAL and/or TAL) described herein and a polynucleotide encoding one or more additional enzymes, wherein the AL and the one or more additional enzymes provide enzymatic activities that promote production of an aromatic compound or improve an aromatic compound manufacturing mixture. In some embodiments, the additional enzyme is 4-coumarate-CoA ligase (4CL), very-long-chain enoyl-CoA reductase (TSC13), chalcone synthase (CHS), 3-hydroxylase (CH3H), O-methyltransferase (OMT), UDP-glucuronosyltransferase (UGT), 4-coumarate 3-hydroxylase, feruloyl-CoA synthetase (FCS), enoyl-CoA hydratase (ECH), benzalacetone synthase (BAS), raspberry ketone/zingerone synthase (RZS1), p-coumaric acid/cinnamic acid carboxyl methyltransferase (CCMT), chalcone isomerase (CHI), and/or 1,2-rhamnosyltransferase.

### Methods

In some aspects, the disclosure provides methods of using host cells for producing products of interest. In some embodiments, the disclosure provides a method comprising culturing a host cell described in this application (e.g., a host cell comprising a heterologous polynucleotide encoding an AL (e.g., a PAL and/or TAL). Methods for culturing cells are described elsewhere in this application. In some embodiments, the disclosure provides a method of producing trans-cinnamic acid from phenylalanine and/or degrading phenylalanine, comprising culturing a host cell described in this application (e.g., a host cell comprising a heterologous polynucleotide encoding an AL (e.g., a PAL and/or TAL)). In some embodiments, the disclosure provides a method of producing p-coumaric acid from tyrosine and/or degrading tyrosine, comprising culturing a host cell described in this application (e.g., a host cell comprising a heterologous polynucleotide encoding an AL (e.g., a PAL and/or TAL)). In some embodiments, the production occurs ex vivo, e.g., in an in vitro cell culture environment. Compositions, cells, enzymes, and methods described in this application are also applicable to industrial settings, including any application wherein there is a need for increased biosynthesis of trans-cinnamic acid and/or p-coumaric acid.

In some embodiments, methods associated with the disclosure include methods of producing one or more of the following products: caffeate, caffeic acid, methyl caffeic acid, ferulic acid, hesperetin, HDG, hydroxybenzalacetone, methyl cinnamate, naringenin, naringin, narirutin, phloretin, phloridzin, raspberry ketone, vanillic acid, vanillin, liquiritgenin, (2S)-flavanone, 2-hydroxy-flavanone, 7,4′-dihydroxyflavanone, 2-hydroxy-isoflavanone, formononetin, biochanin, 2′-hydroxy-formononetin, 4-coumaroyl-CoA, apigenin, chalconaringenin, daidzein, daidzin, malonyldaidzein (MGD), dihydrodaidzein, dihydrodaidzein-sulfate, O-desmethylangolensin, 6-OH—O-desmethylangolensin, tetrahydrodaidzein, equol, equol-7-glucuronide, equol-4′-sulfate, 5-hydroxy equol, hippuric acid, 4-hydroxybenzoic acid, 2,6-dimethoxy benzoic acid, fumaric acid, 4-ethylphenol, glutaric acid, 2-phenylpropionic acid, gallic acid, resorcinolsulfate, disometin, chrysoeriol, chrysoeriol-4′-glucuronide, chrysoeriol-7-glucuronide, coumestrol, eriodictyol, dihydroquercetin, genistein, genistin, malonylgenistin (MGG), glycitein, isorhamnetin, kaempferol, laricitrin, luteolin, luteolin-3′-glucuronide, luteolin-4′-glucuronide, morin, myricetin, tetramethylated myricetin, 3,5-dihydroxyphenylacetic acid, 3,4,5-trihydroxyphenylacetic acid, methylated myricetin, myricetin monoglucuronide, myricetin diglucuronide, dimethylated myricetin, pentahydroxy-flavanone, dihydromyricetin, 2R,3S,4S-flavan-3-ol, (+)-Afzelechin, (+)-catechin, (+)-galocatechin, proanthocyanidin, (−)-epiafzelechin, (−)-eoicatechin, (−)-epigallocatechin, taxifolin, dihydroquercetin, aromadendrin, dihydrokaempferol, dihydroquercetin, dihydroflavonol, quercetin, isoquercetin, rutin, peonidin, syringetin, tetrahydroxychalcone, trangeretin, chalcone, 6′-deoxychalcone, isoliquiritigenin, tetraketide, DHK, leuco-pelargonidin, pelargonidin, a pelargonidin-based anthocyanin, DHQ, leuco-cyanidin, cyanidin, a cyanidin-based anthocyanin, DHM, leuco-delphinidin, delphinidin, a delphidin-based anthocyanin, petunidin, malvidin, flavonol, flavone, flavanone, isoflavone, isoflavanone, anthocyanin, cinnamate, methylcinnamate, cinnamoyl-CoA, cinnamaldehyde, styrene, pinocembrin chalcone, pinocembrin, chrysin, baicalein, curcumin, and/or bismethoxy curcumin, or derivatives thereof.

In some aspects, the disclosure provides a method of producing aromatic compounds for use in the fragrance and/or flavor industries. For example, trans-cinnamic acid has a honey-like odor and can be used to impart cinnamon-like flavors, while p-coumaric acid is found in many natural foods and beverages. In some embodiments, trans-cinnamic acid and/or p-coumaric acid are intermediates produced as part of a method for producing an aromatic compound. The disclosure is directed, in part, to methods of producing an aromatic compound using an AL (e.g., a PAL and/or TAL) described in this disclosure, or a nucleic acid encoding the same, or a host cell comprising any thereof.

In some embodiments, an AL is engineered to produce increased titers of trans-cinnamate as a first step of producing hesperetin dihydrochalcone 4′-O-glucoside (HDG). In some embodiments, an AL is engineered to produce increased titers of p-coumarate as a first step of producing hesperetin dihydrochalcone 4′-O-glucoside (HDG). HDG is a flavonone that may be used as a sweetener. Without wishing to be bound by any theory, it is believed that increased titers of HDG can be produced by increasing production of trans-cinnamate or p-coumarate. p-coumarate produced by a TAL or converted from trans-cinnamate produced by a PAL is a substrate for 4-coumarate-CoA ligase (4CL), which produces p-coumaroyl CoA from p-coumarate. p-coumaroyl CoA is converted to dihydrocoumaroyl-CoA by very-long-chain enoyl-CoA reductase (TSC13) and then to phloretin by chalcone synthase (CHS). Phloretin is converted to 3-hydroxyphloretin by chalcone 3-hydroxylase (CH₃H), then to hesperetin dihydrochalcone by O-methyltransferase. Finally, hesperetin dihydrochalcone is converted to HDG by a UDP-glucuronosyltransferase (UGT). In some embodiments, a host cell expressing an AL also comprises any one of the enzymes required to produce HDG from trans-cinnamate and/or p-coumarate.

In some embodiments, an AL is engineered to produce increased titers of trans-cinnamate as a first step of producing ferulic acid. In some embodiments, an AL is engineered to produce increased titers of p-coumarate as a first step of producing ferulic acid. Ferulic acid is a hydroxycinnamic acid that may be used in various foods or fragrances. Without wishing to be bound by any theory, it is believed that increased titers of ferulic acid can be produced by increasing production of trans-cinnamate or p-coumarate. p-coumarate produced by a TAL or converted from trans-cinnamate produced by a PAL is a substrate for 4-coumarate 3-hydroxylase, which produces caffeic acid from p-coumarate. Caffeic acid is then converted to ferulic acid by an O-methyltransferase enzyme. In some embodiments, a host cell expressing an AL also comprises any one of the enzymes required to produce ferulic acid from trans-cinnamate and/or p-coumarate.

In some embodiments, an AL is engineered to produce increased titers of trans-cinnamate as a first step of producing vanillin. In some embodiments, an AL is engineered to produce increased titers of p-coumarate as a first step of producing vanillin. Vanillin is a major component of vanilla. Without wishing to be bound by any theory, it is believed that increased titers of vanillin can be produced by increasing production of trans-cinnamate or p-coumarate. p-coumarate produced by a TAL or converted from trans-cinnamate produced by a PAL is a substrate for 4-coumarate 3-hydroxylase, which produces caffeic acid from p-coumarate. Caffeic acid is then converted to ferulic acid by an O-methyltransferase enzyme. Ferulic acid is then converted to feruloyl-CoA by feruloyl-CoA synthetase (FCS), and finally to vanillin by enoyl-CoA hydratase (ECH). In some embodiments, a host cell expressing an AL also comprises any one of the enzymes required to produce vanillin from trans-cinnamate and/or p-coumarate.

In some embodiments, an AL is engineered to produce increased titers of trans-cinnamate as a first step of producing raspberry ketone. In some embodiments, an AL is engineered to produce increased titers of p-coumarate as a first step of producing raspberry ketone. Raspberry ketone is a phenolic compound that is the primary aroma compound of red raspberries. Without wishing to be bound by any theory, it is believed that increased titers of raspberry ketone can be produced by increasing production of trans-cinnamate or p-coumarate. p-coumarate produced by a TAL or converted from trans-cinnamate produced by a PAL is a substrate for 4-coumarate-CoA ligase (4CL), which produces p-coumaroyl CoA from p-coumarate. p-coumaroyl CoA is converted to 4-hydroxybenzildene acetone by benzalacetone synthase (BAS), then to raspberry ketone by raspberry ketone/zingerone synthase (RZS1). In some embodiments, a host cell expressing an AL also comprises any one of the enzymes required to produce raspberry ketone from trans-cinnamate and/or p-coumarate.

In some embodiments, an AL is engineered to produce increased titers of trans-cinnamate as a first step of producing methyl cinnamate. In some embodiments, an AL is engineered to produce increased titers of p-coumarate as a first step of producing methyl cinnamate. Methyl cinnamate is a methyl ester of cinnamic acid. Methyl cinnamate is used as a flavor or fragrance as its flavor is fruity and strawberry-like and its aroma is sweet and fruity with hints of cinnamon and strawberry. Without wishing to be bound by any theory, it is believed that increased titers of methyl cinnamate can be produced by increasing production of trans-cinnamate or p-coumarate. p-coumarate produced by a TAL or converted from trans-cinnamate produced by a PAL is a substrate for a p-coumaric acid/cinnamic acid carboxyl methyltransferase (CCMT), which produces methyl cinnamate. In some embodiments, a host cell expressing an AL also comprises any one of the enzymes required to produce methyl cinnamate from trans-cinnamate and/or p-coumarate.

In some embodiments, an AL is engineered to produce increased titers of trans-cinnamate as a first step of producing naringin. In some embodiments, an AL is engineered to produce increased titers of p-coumarate as a first step of producing naringin. Naringin is a flavonone found naturally in many citrus fruits. In grapefruit, naringin is responsible for the fruit's bitter tase. Without wishing to be bound by any theory, it is believed that increased titers of naringin can be produced by increasing production of trans-cinnamate or p-coumarate. p-coumarate produced by a TAL or converted from trans-cinnamate produced by a PAL is a substrate for 4-coumarate-CoA ligase (4CL), which produces p-coumaroyl CoA from p-coumarate. p-coumaroyl CoA is converted to naringenin chalcone by chalcone synthase (CHS), then to naringenin by chalcone isomerase (CHI). Naringenin is converted to prunin by flavonone 7-O-glucosyltransferase, which is then converted to naringin by 1,2-rhamnosyltransferase. In some embodiments, a host cell expressing an AL also comprises any one of the enzyme required to produce naringin from trans-cinnamate and/or p-coumarate.

In some embodiments, a method comprises converting one or more substrates into one or more aromatic compounds. In some embodiments, a method converts a sugar (e.g., glucose) into one or more aromatic compounds, e.g., by a plurality of steps comprising L-phenylalanine and/or L-tyrosine as intermediates. In some embodiments, L-phenylalanine and/or L-tyrosine are substrates for the production of aromatic compounds. In some embodiments, the disclosure provides a method of converting L-phenylalanine and/or L-tyrosine to trans-cinnamic acid and/or p-coumaric acid by contacting L-phenylalanine and/or L-tyrosine with any host cell described in this disclosure. In some embodiments, the method further comprises converting trans-cinnamic acid and/or p-coumaric acid into a downstream product to produce an aromatic compound. In some embodiments, converting trans-cinnamic acid and/or p-coumaric acid into a downstream product comprises contacting the trans-cinnamic acid and/or p-coumaric acid with an enzyme, e.g., a recombinant enzyme, e.g., of the shikimate pathway. In some embodiments, the enzyme, e.g., a recombinant enzyme, e.g., of the shikimate pathway is within a host cell, e.g., a host cell comprising the AL, e.g., the PAL and/or TAL.

The disclosure is also directed to a method for improving an aromatic compound manufacturing mixture comprising contacting an aromatic compound manufacturing mixture with an AL (e.g., a PAL and/or TAL), a nucleic acid encoding either thereof, or a host cell comprising any thereof. As used in this disclosure, the term “aromatic compound manufacturing mixture” refers to a mixture comprising a plurality of metabolic intermediates, input materials, and/or manufacturing reagents. Optionally, an aromatic compound manufacturing mixture comprises one or more aromatic compounds. In some embodiments, an aromatic compound manufacturing mixture can be improved, where improved means increasing the level of a desired metabolic intermediate or aromatic compound, or decreasing the level of an undesirable metabolic intermediate or an input material. In some embodiments, improving comprises contacting the mixture with a manufacturing reagent or enzyme (or a composition comprising either thereof, e.g., a cell). For example, an aromatic compound manufacturing mixture may comprise trans-cinnamic acid and/or p-coumaric acid, and optionally one or more metabolic intermediates, input materials, and/or manufacturing reagents. In some embodiments, a method of improving an aromatic compound manufacturing mixture comprises producing an aromatic compound using an AL (e.g., a PAL and/or TAL) described in this disclosure, or a nucleic acid encoding the same, or a host cell comprising any thereof.

In some embodiments, a host cell and/or an AL (e.g., a PAL and/or TAL) comprise one or more modifications to enhance their effectiveness (e.g., activity and/or stability (e.g., half-life)) in a selected mode of biosynthesis. For example, an AL (e.g., a PAL and/or TAL) may comprise a modification that increases stability and/or activity of the enzyme at acidic pH, e.g., to improve the effectiveness of the PAL or TAL when used in an industry-level batch culture. In some embodiments, the PAL or TAL is immobilized to another agent, e.g., a different enzyme, a polymer (e.g., polysaccharide (e.g., starch)), or an inorganic carrier (e.g., silica gel). Immobilization may increase enzyme stability and/or shelf-life.

### Compositions

Further aspects of the disclosure relate to compositions containing trans-cinnamic acid and/or p-coumaric acid. Culturing of host cells associated with the disclosure can result in compositions comprising products, including trans-cinnamic acid and/or p-coumaric acid. In some embodiments, compositions obtained by culturing host cells associated with the disclosure result in compositions in which at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the total products in the composition is/are trans-cinnamic acid and/or p-coumaric acid.

Compositions associated with the disclosure can further comprise additional components as would be understood by one of ordinary skill in the art. For example, it should be appreciated that in some embodiments, compositions comprising trans-cinnamic acid and/or p-coumaric acid can include cell culture fermentation broth or cell culture supernatants. In other embodiments, compositions may include trans-cinnamic acid and/or p-coumaric acid in a form that has been purified from cell culture fermentation broth or cell culture supernatants.

In some embodiments, cells associated with the invention are cultured in the presence of an organic solvent overlay. As used in this disclosure, an organic solvent overlay refers to a layer comprising one or more organic solvents that is added to a cell culture sample. The organic solvent overlay may partially or fully cover the cell culture sample. The use of an organic solvent overlay can assist with reducing or alleviating host cell toxicity caused by increased concentrations of products. In some embodiments, compositions comprising trans-cinnamic acid and/or p-coumaric acid further comprise one or more components of an organic solvent overlay (e.g., dodecane).

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

In order that the invention described in the present application may be more fully understood, the following examples are set forth. The examples described in this application are offered to illustrate the systems and methods provided in this disclosure and are not to be construed in any way as limiting their scope.

### Example 1. Identification of Variant ALs that Produce Increased Trans-Cinnamic Acid

This Example describes the identification of variant aromatic amino acid ammonia lyases (ALs) that have phenylalanine ammonia lyase (PAL) activity and are capable of producing increased amounts of trans-cinnamic acid relative to that produced by the wild type PAL from Anabaena variabilis (AvPAL; UniProKB Accession No. Q3M5Z3; SEQ ID NO: 1).

To identify variant ALs capable of producing increased amounts of trans-cinnamic acid relative to AvPAL, a first protein engineering library of approximately 584 variant ALs and a second protein engineering library of approximately 4000 variant ALs were generated based on the AvPAL sequence (SEQ ID NO: 1). The variant ALs within the libraries comprised amino acid substitutions at one or more amino acid residues including the following seven amino acid residues within the AvPAL sequence (SEQ ID NO: 1): T102, L104, F107, L108, G218, L219, and M222.

The first protein engineering library of approximately 584 variant ALs was transformed into DH5a competent E. coli cells and stored at −80° C. in glycerol. To initiate cell growth in preparation for screening, glycerol stocks of the AL variant transformants were inoculated into LB media containing 100 μg/mL of carbenicillin and shaken at 1,000 rpm overnight at 37° C. After the initial growth phase, 10 μL of each overnight culture was inoculated into fresh 990 μL LB media containing 100 μg/mL of carbenicillin. The transformants were shaken at 1,000 rpm at 37° C. for two hours, followed by addition of IPTG at a final concentration of 0.2 μL/mL. The transformants were further shaken at 1,000 rpm for four hours at 37° C., then centrifuged at 4,000×g for ten minutes. The supernatant was discarded and the cell pellets were resuspended in phosphate-buffered saline (PBS; 500 mM, pH 7.4).

The AL variants were evaluated for PAL activity in triplicate in a primary screen using a whole-cell assay. 20 μL of the variant AL transformants in PBS was added to 500 L of M9 media containing phenylalanine (40 mM). After a one hour incubation, the solution was centrifuged and 50 μL of the supernatant was transferred to 50 μL of M9 media for analysis. The solution was analyzed for absorbance at 290 nM, a wavelength at which trans-cinnamic acid absorbs. The wild-type AvPAL and an AvPAL mutant comprising a G218A amino acid substitution were included as controls.

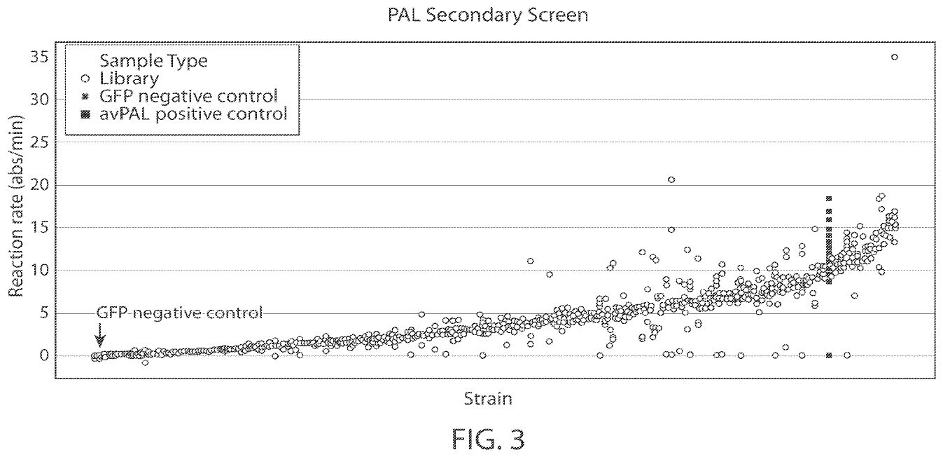

The 300 variant ALs with the highest PAL activity in the primary screen were analyzed further in a secondary screen to confirm PAL activity in host cell lysates. Variant AL transformants were prepared using the methods described above for the primary screen, but instead of resuspending the cell pellets in PBS, the cell pellets were resuspended in 125 μL of lysis buffer (1× Bugbuster lysis reagent, 2.5 mM 1,4-Dithiothreitol (DTT), 0.2 mM Phenylmethylsulfonyl fluoride (PMSF), 3 U/μL rLysozyme, 0.0025 U/μL Benzonase Nuclease). The lysed pellets were added to 96-well plates, and continuous, kinetic absorbance measurements were collected at 290 nm. Measurements were taken over ten minutes while the 96-well plates were shaken in a slow, orbital movement at 28° C. Results are shown in FIG. 3.

Variant ALs with the highest PAL activity as observed in the secondary screen are shown in Table 3. A strain expressing the wild-type AvPAL (t888841) was included as a positive control. A strain expressing GFP was included as a negative control. The secondary screen activity scores were calculated by Z-score, normalizing each experimental value to the value of the wild-type control. Overall, 24 variant ALs produced an activity score greater than 1.00. Strain t900097 showed the highest improvement over the control strains, with an activity score of 1.79.

Without wishing to be bound by any theory, the amino acid substitutions in these 24 variant ALs may affect the substrate binding site of the enzyme by influencing its shape and chemical composition, which may produce changes in substrate binding affinity and/or enzymatic catalysis.

TABLE 3
Trans-cinnamic acid production by variant ALs

| | | | Secondary Screen |
|---|---|---|---|
| | | | Activity Score |
| | AL | | (normalized to the |
| | protein | | average value of the |
| | SEQ ID | Substitutions Relative | WT control |
| Strain ID | NO: | to SEQ ID NO: 1 | (t888841)) |
| t888841 | 1 | N/A | N/A |
| (wild-type) | | | |
| GFP | N/A | N/A | N/A |
| Negative | | | |
| Control | | | |
| t900097 | 5 | T102H; L104M; G218A | 1.79 |
| t900270 | 6 | L104M; L108T; G218A | 1.51 |
| t900424 | 7 | T102K | 1.48 |
| t902826 | 8 | T102E; L104M; L108T; | 1.38 |
| | | G218A; M222L | |
| t900166 | 9 | T102S; M222L | 1.36 |
| t903111 | 10 | T102H; L104M; L219I | 1.35 |
| t903984 | 11 | T102H; L104M; L108T; | 1.33 |
| | | G218A; M222V | |
| t899644 | 12 | T102K; G218A | 1.30 |
| t903141 | 13 | T102S; L108T; G218S; | 1.28 |
| | | M222L | |
| t899646 | 14 | T102E; L108T; M222I | 1.17 |
| t900622 | 15 | T102E; G218S | 1.16 |
| t904262 | 16 | T102K; L104I; L108T; | 1.16 |
| | | M222L | |
| t899973 | 17 | T102S; L104M; L108M | 1.15 |
| t900177 | 18 | T102K; G218A; M222T | 1.14 |
| t902547 | 19 | T102S; L104M; L219I; | 1.11 |
| | | M222L | |
| t899454 | 20 | T102H; L108T | 1.10 |
| t900320 | 21 | L104M; M222V | 1.10 |
| t904103 | 22 | T102H; L104M; G218A; | 1.09 |
| | | M222T | |
| t900578 | 23 | M222L | 1.07 |
| t899285 | 24 | T102S; L108T; M222L | 1.04 |
| t903447 | 25 | T102S; L108V; G218A | 1.04 |
| t902439 | 26 | L104A; L108T; G218A | 1.03 |
| t904333 | 27 | L104V; L108T | 1.03 |
| t903300 | 28 | T102K; L108V; M222L | 1.00 |

### Example 2. Identification of Variant ALs that Exhibit Tyrosine Ammonia Lyase Activity

AL enzymes can also exhibit tyrosine ammonia lyase (TAL) activity. ALs are often promiscuous in terms of enzymatic activity, allowing ALs to be active on L-phenylalanine, L-tyrosine, and/or L-histidine as substrates. As described in the present disclosure, amino acid substitutions at specific positions (e.g., F107 and/or L108) may shift the AL binding affinity from one substrate to another. This Example describes the engineering of the AvPAL parent enzyme at specific amino acid residues to shift its affinity from one substrate (e.g., L-phenylalanine) to another substrate (e.g., L-tyrosine).

In order to assess whether any of the variant ALs identified in Example 1 also exhibit TAL activity, the second, 4000-member protein engineering library described in Example 1 was also screened for TAL activity by assessing whether the AL variants were capable of producing increased amounts of p-coumaric acid relative to AvPAL on a tyrosine substrate.

The AL variants were evaluated for TAL activity in triplicate in a primary screen using a whole-cell assay. 20 μL of the variant AL transformants in PBS was added to 500 μL of M9 media containing tyrosine (40 mM). After a one hour incubation, the solution was centrifuged and 50 μL of the supernatant was transferred to 50 μL of M9 media for analysis. The solution was analyzed for absorbance at 310 nm and 600 nm. The wild-type AvPAL and a TAL (RsTAL) were included as positive controls. A strain expressing GFP was included as a negative control.

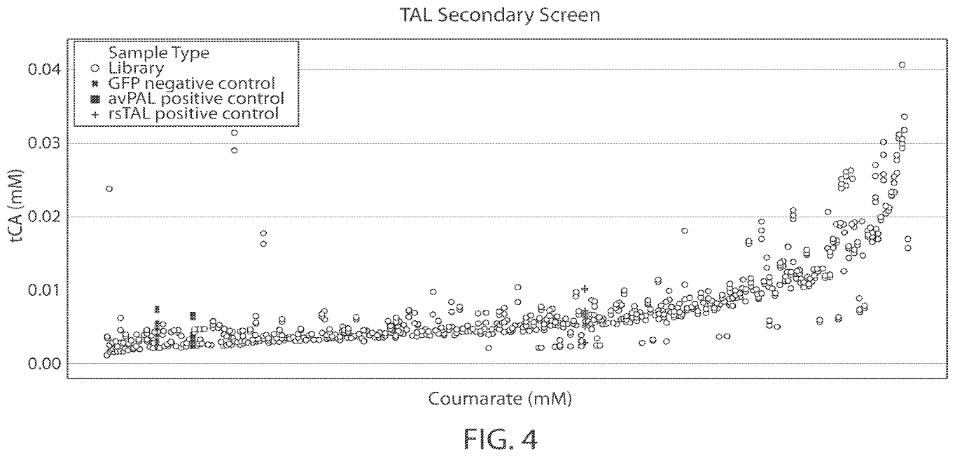

The 300 variant ALs with the highest TAL activity in this primary screen were analyzed further in a secondary screen using cell lysates to confirm TAL activity. To prepare the cell lysates, variant AL transformants were prepared as described for the primary screen in Example 1, but instead of resuspending the cell pellets in PBS, the cell pellets were resuspended in 250 μL of lysis buffer (1× Bugbuster lysis reagent, 2.5 mM 1,4-Dithiothreitol (DTT), 0.2 mM Phenylmethylsulfonyl fluoride (PMSF), 3 U/μL rLysozyme, 0.0025 U/μL Benzonase Nuclease). The cell pellets were lysed and centrifuged at 4,000×g for 3 minutes. 50 μL of clarified lysate from each sample was added to a well of an assay plate containing 150 μL of assay buffer (1 mM L-tyrosine in M9 media) per well. After 4 hours of incubation time at room temperature, the assay plates containing the lysates and assay buffer were read at 290 nm, 310 nm, and 600 nm. Results are shown in FIG. 4.

Variant ALs with the highest TAL activity as observed in the secondary screen using the cell lysate assay are shown in Table 4. The secondary screen activity scores were calculated by Z-score, normalizing each experimental value to the value of the RsTAL Control (strain t915919). Overall, 167 variant ALs produced an activity score greater than 1.00. Strain t900309 showed the highest improvement over the control strains, with an activity score of 3.82.

Without wishing to be bound by any theory, the amino acid substitutions in these 167 variant ALs may affect the substrate binding site of the enzyme by influencing its shape and chemical composition, which may produce changes in substrate binding affinity and/or enzymatic catalysis.

TABLE 4
p-coumaric acid production by variant ALs

| | | | Secondary Screen |
|---|---|---|---|
| | | | Activity Score |
| | AL | | (normalized to the |
| | protein | | average value of |
| | SEQ | Substitutions Relative | the RsTAL control |
| Strain ID | ID NO: | to SEQ ID NO: 1 | (t915919)) |
| t888841 | 1 | N/A | N/A |
| (wild-type) | | | |
| GFP | N/A | N/A | N/A |
| Negative | | | |
| Control | | | |
| t915919 | 4 | RsTAL Control | N/A |
| t900309 | 29 | L104A; L108Q; L219I; | 3.82 |
| | | M222N | |
| t904359 | 30 | T102S; L108Q; G218A | 3.65 |
| | | L219I | |
| t899349 | 31 | T102H; L104M; L108M; | 3.65 |
| | | L219I; M222L | |
| t904360 | 32 | T102E; F107Y; L108M; | 3.57 |
| | | G218S; L219I; M222N | |
| t900541 | 33 | L104I; L108H; G218A; | 3.52 |
| | | L219I; M222V | |
| t903485 | 34 | T102E; L104M; F107Y; | 3.47 |
| | | M222I | |
| t903905 | 35 | T102E; L104V; F107Y; | 3.46 |
| | | L108M; L219I; M222T | |
| t900376 | 36 | T102S; L104I; G218S; | 3.46 |
| | | L219I; M222V | |
| t900070 | 37 | L104V; G218A; M222L | 3.45 |
| t903327 | 38 | T102K; L108H; G218A; | 3.40 |
| | | L219I; M222T | |
| t903331 | 39 | L1041; L108M; G218S | 3.36 |
| t899517 | 40 | T102H; F107Y; L108M; | 3.36 |
| | | L219I; M222V | |
| t902928 | 41 | L104V; F107H; L108Q; | 3.35 |
| | | M222L | |
| t903870 | 42 | T102K; L104A; L108Q; | 3.32 |
| | | G218A; L219I | |
| t900570 | 43 | T102S; L104A; F107S; | 3.31 |
| | | L219I; M222N | |
| t901073 | 44 | T102S; L108H; G218S; | 3.29 |
| | | M222V | |
| t900842 | 45 | T102K; L104A; L108H; | 3.29 |
| | | L219I; M222N | |
| t903554 | 46 | T102S; L108H; M222N | 3.27 |
| t902525 | 47 | T102H; L104M; L108M; | 3.25 |
| | | L219I | |
| t900314 | 48 | T102K; L104A; F107Y; | 3.24 |
| | | L108V; G218A; L219I; | |
| | | M222N | |
| t903000 | 49 | T102H; L108M; G218S; | 3.23 |
| | | M222L | |
| t900137 | 50 | T102E; L104M; F107Y; | 3.22 |
| | | L108M; G218A; L219I | |
| t902777 | 51 | T102E; L104V; F107H; | 3.21 |
| | | M222N | |
| t900470 | 52 | T102H; F107H; L108M; | 3.18 |
| | | L219I; M222T | |
| t903261 | 53 | T102H; L104V; F107S; | 3.15 |
| | | L108Q; G218S; M222T | |
| t899659 | 54 | T102E; L104M; F107S; | 3.14 |
| | | L108M; G218A; L219I | |
| t903839 | 55 | T102E; L104V; F107Y; | 3.14 |
| | | L108M; L219I | |
| t903683 | 56 | T102K; F107Y; L108V; | 3.09 |
| | | L219I; M222V | |
| t903786 | 57 | T102E; L104M; F107H; | 3.07 |
| | | G218A; L219I; M222L | |
| t900534 | 58 | T102H; L104M; F107Y; | 3.07 |
| | | G218A; M222I | |
| t899681 | 59 | F107Y; L219I; M222N | 3.06 |
| t899385 | 60 | T102S; L104A; F107Y; | 3.06 |
| | | L108M | |
| t899234 | 61 | T102E; F107Y; L108M; | 3.05 |
| | | G218A; L219I; M222V | |
| t903684 | 62 | T102K; L104M; F107Y; | 3.03 |
| | | L108V; G218S; M222N | |
| t902742 | 63 | L104I; F107H; L108M; | 3.02 |
| | | L219I; M222V | |
| t901124 | 64 | T102K; F107Y; L108V; | 3.00 |
| | | M222L | |
| t903556 | 65 | T102E; L104I; F107S; | 2.97 |
| | | L108T; L219I; M222I | |
| t900261 | 66 | L104I; F107H; L219I | 2.97 |
| t903832 | 67 | T102S; L104I; F107S; | 2.95 |
| | | G218S; L219I; M222V | |
| t899661 | 68 | T102H; L104M; F107Y; | 2.94 |
| | | L108T; G218S; M222L | |
| t900441 | 69 | L104M; F107Y; L108T; | 2.92 |
| | | M222L | |
| t904233 | 70 | T102E; F107Y; L108M; | 2.90 |
| | | M222T | |
| t902516 | 71 | T102S; L104A; F107Y; | 2.90 |
| | | L108T; G218S; L219I | |
| t900250 | 72 | T102K; L104I; F107H; | 2.90 |
| | | L219I; M222I | |
| t902538 | 73 | T102K; L104I; F107H; | 2.88 |
| | | L108H; M222T | |
| t900135 | 74 | T102K; L104I; F107H; | 2.86 |
| | | L219I; M222L | |
| t904309 | 75 | T102H; L104A; F107Y; | 2.86 |
| | | L108T; M222T | |
| t899303 | 76 | T102K; F107H; L108M; | 2.84 |
| | | L219I; M222I | |
| t903599 | 77 | T102H; L104M; F107S; | 2.82 |
| | | L108M; G218A; M222I | |
| t900257 | 78 | T102S; L104M; L108Q; | 2.82 |
| | | G218A; M222V | |
| t900204 | 79 | T102E; L104V; L108M; | 2.81 |
| | | M222I | |
| t903569 | 80 | F107Y; L108V; M222V | 2.79 |
| t903678 | 81 | L104A; F107H; L108V; | 2.78 |
| | | G218A; M222T | |
| t900518 | 82 | T102K; L104A; F107H; | 2.77 |
| | | L108T; G218A | |
| t899485 | 83 | L108M; G218S; M222I | 2.76 |
| t903247 | 84 | L104V; F107H; L108H; | 2.75 |
| | | M222L | |
| t902757 | 85 | T102H; F107H; G218A; | 2.74 |
| | | L219I; M222V | |
| t902788 | 86 | T102E; L104V; F107H; | 2.73 |
| | | L108H; M222I | |
| t899939 | 87 | L104A; L108M; L219I; | 2.73 |
| | | M222N | |
| t903070 | 88 | T102K; F107Y; L219I; | 2.72 |
| | | M222I | |
| t901084 | 89 | F107S; L108Q; L219I; | 2.69 |
| | | M222V | |
| t900075 | 90 | T102E; L104I; F107Y; | 2.68 |
| | | L108H; M222N | |
| t899817 | 91 | T102H; F107Y; L108M; | 2.66 |
| | | M222L | |
| t900304 | 92 | T102E; L104V; F107Y; | 2.65 |
| | | L108H; L219I; M222T | |
| t903671 | 93 | T102E; F107Y; G218S; | 2.64 |
| | | L219I; M222V | |
| t899801 | 94 | F107S; L108H; G218S; | 2.63 |
| | | L219I; M222L | |
| t903810 | 95 | T102E; L104V; F107H; | 2.63 |
| | | L219I; M222V | |
| t903609 | 96 | L104A; L108V; G218A; | 2.62 |
| | | L219I; M222L | |
| t904224 | 97 | T102E; L104I; L108M; | 2.62 |
| | | M222I | |
| t899897 | 98 | T102H; L104V; L108H; | 2.61 |
| | | L219I; M222L | |
| t904382 | 99 | T102H; L104V; L108H; | 2.60 |
| | | M222I | |
| t899614 | 100 | T102E; F107H; L108H; | 2.60 |
| | | G218A; L219I | |
| t900493 | 101 | T102E; F107Y; G218S; | 2.60 |
| | | M222V | |
| t900056 | 102 | T102S; L108M; L219I; | 2.60 |
| | | M222I | |
| t902703 | 103 | T102S; F107H; G218A; | 2.59 |
| | | M222T | |
| t903478 | 104 | L104A; F107H; L219I; | 2.58 |
| | | M222I | |
| t903415 | 105 | T102S; F107Y; L219I; | 2.57 |
| | | M222L | |
| t900280 | 106 | T102E; F107Y; L108M; | 2.57 |
| | | G218A; M222V | |
| t904001 | 107 | T102E; L104M; F107S; | 2.56 |
| | | L108T; G218S; L219I; | |
| | | M222V | |
| t897903 | 108 | T102S; L104V; F107H; | 2.54 |
| | | L108H | |
| t900237 | 109 | T102E; F107Y; L108M; | 2.54 |
| | | L219I; M222I | |
| t903852 | 110 | T102K; F107S; L108H; | 2.53 |
| | | G218A; L219I; M222L | |
| t903783 | 111 | T102K; L104A; F107Y; | 2.53 |
| | | L108T; G218S; L219I; | |
| | | M222L | |
| t902507 | 112 | T102E; L104M; F107H; | 2.51 |
| | | G218S; M222V | |
| t903472 | 113 | T102S; L104V; F107S; | 2.49 |
| | | L108H; L219I; M222I | |
| t903445 | 114 | T102E; L104M; L108M; | 2.48 |
| | | G218A; L219I; M222T | |
| t899874 | 115 | T102E; L104V; F107Y; | 2.46 |
| | | L108H; G218S | |
| t903669 | 116 | T102H; L104V; F107S; | 2.45 |
| | | L108T; G218A; M222T | |
| t899582 | 117 | T102S; F107H; L219I; | 2.40 |
| | | M222I | |
| t903313 | 118 | T102S; L104A; F107H; | 2.40 |
| | | M222L | |
| t903018 | 119 | T102K; L104M; F107Y; | 2.39 |
| | | L108H; G218A; M222I | |
| t904043 | 120 | T102E; L104A; L108M; | 2.38 |
| | | M222V | |
| t899778 | 121 | T102H; L104V; F107H; | 2.37 |
| | | L108T; M222I | |
| t902749 | 122 | T102E; F107Y; L108V; | 2.36 |
| | | M222V | |
| t903837 | 123 | T102E; L104A; F107Y; | 2.36 |
| | | M222N | |
| t899595 | 124 | T102E; F107S; L108T; | 2.35 |
| | | G218A; L219I; M222I | |
| t903999 | 125 | T102H; L104A; F107H; | 2.35 |
| | | L108T; G218S; L219I; | |
| | | M222L | |
| t899254 | 126 | T102H; L104M; F107H; | 2.34 |
| | | L219I; M222L | |
| t899587 | 127 | T102E; F107Y; L108M; | 2.34 |
| | | G218A; M222I | |
| t903126 | 128 | T102E; L104M; F107H; | 2.33 |
| | | L108V; L219I; M222L | |
| t897909 | 129 | T102H; F107Y; L108H; | 2.32 |
| | | G218S; M222L | |
| t899316 | 130 | L104M; L108M; L219I; | 2.32 |
| | | M222L | |
| t903927 | 131 | L104I; F107Y; L108H; | 2.31 |
| | | G218A; M222V | |
| t899276 | 132 | T102E; L104M; F107S; | 2.28 |
| | | G218A; L219I; M222N | |
| t903921 | 133 | T102E; L104A; F107H; | 2.27 |
| | | M222I | |
| t899696 | 134 | L104V; F107Y; L108H; | 2.27 |
| | | G218A; M222I | |
| t899835 | 135 | T102E; L104I; F107S; | 2.25 |
| | | L108H; L219I; M222L | |
| t900002 | 136 | T102K; L104M; L108M; | 2.25 |
| | | G218A | |
| t897927 | 137 | T102E; F107Y; L108T; | 2.24 |
| | | G218A; L219I; M222I | |
| t902820 | 138 | T102S; L1041; F107H; | 2.23 |
| | | L108V; M222L | |
| t899604 | 139 | T102K; L108M; G218A; | 2.22 |
| | | L219I; M222L | |
| t899513 | 140 | T102S; F107S; L108H; | 2.21 |
| | | G218S; M222V | |
| t903230 | 141 | T102H; L108H; L219I; | 2.18 |
| | | M222L | |
| t900395 | 142 | T102S; L104A; F107H; | 2.18 |
| | | L108V; M222L | |
| t904192 | 143 | T102E; L104A; F107H; | 2.17 |
| | | G218A; M222I | |
| t899840 | 144 | T102E; L104V; L108M; | 2.16 |
| | | M222L | |
| t904358 | 145 | T102E; L104A; F107H; | 2.14 |
| | | M222V | |
| t899871 | 146 | T102H; F107Y; L108H; | 2.13 |
| | | G218A; L219I; M222I | |
| t902832 | 147 | T102E; F107S; L108V; | 2.12 |
| | | M222L | |
| t899261 | 148 | T102H; L108M; L219I; | 2.09 |
| | | M222V | |
| t903895 | 149 | T102S; L104M; F107S; | 2.08 |
| | | L108H; G218A; L219I; | |
| | | M222V | |
| t902834 | 150 | T102H; L1041; F107H; | 2.07 |
| | | L108T; M222T | |
| t899266 | 151 | T102E; F107S; G218A; | 2.03 |
| | | M222N | |
| t902888 | 152 | T102H; L104V; F107Y; | 2.02 |
| | | L108H; L219I; M222V | |
| t903908 | 153 | T102E; F107Y; L108M; | 2.00 |
| | | L219I; M222V | |
| t899507 | 154 | T102H; L104I; F107H; | 1.97 |
| | | L219I; M222V | |
| t900251 | 155 | T102K; L104A; F107H; | 1.96 |
| | | L108V; G218A | |
| t903988 | 156 | T102K; L104M; F107H; | 1.95 |
| | | G218A; M222T | |
| t902587 | 157 | T102S; F107H; L108H; | 1.95 |
| | | M222V | |
| t900125 | 158 | L104A; L108M; G218A | 1.94 |
| t900247 | 159 | L104V; L108M | 1.93 |
| t899718 | 160 | T102H; L104M; L108H; | 1.89 |
| | | G218A; L219I; M222L | |
| t899788 | 161 | T102H; L108M; G218A; | 1.89 |
| | | M222T | |
| t900451 | 162 | T102H; L104I; F107H; | 1.83 |
| | | M222I | |
| t899501 | 163 | T102E; F107H; L108M; | 1.78 |
| | | L219I; M222V | |
| t897929 | 164 | F107S; L108T; G218A; | 1.78 |
| | | L219I; M222I | |
| t904107 | 165 | T102E; L104V; F107Y; | 1.77 |
| | | L108H; L219I; M222L | |
| t900001 | 166 | T102K; L104V; F107H; | 1.72 |
| | | M222I | |
| t899786 | 167 | T102H; L104M; F107Y; | 1.72 |
| | | L108H; G218A; L219I; | |
| | | M222L | |
| t902438 | 168 | T102E; F107Y; L108V; | 1.71 |
| | | G218S; M222N | |
| t902554 | 169 | T102E; L104V; L108H; | 1.69 |
| | | L219I; M222I | |
| t902740 | 170 | L104A; F107H; L108T; | 1.69 |
| | | G218A; M222I | |
| t900090 | 171 | T102E; L104I; F107H; | 1.69 |
| | | L108V; M222I | |
| t902435 | 172 | L104V; L108H; L219I; | 1.65 |
| | | M222I | |
| t904324 | 173 | T102E; L104V; F107H; | 1.65 |
| | | M222I | |
| t903946 | 174 | L108M; G218A; L219I; | 1.62 |
| | | M222V | |
| t900007 | 175 | T102H; L108M; G218A; | 1.61 |
| | | L219I; M222L | |
| t897910 | 176 | T102S; L104M; F107H; | 1.59 |
| | | M222T | |
| t897898 | 177 | T102E; L104V; F107Y; | 1.58 |
| | | L108H; G218A; M222I | |
| t900300 | 178 | T102E; L104I; F107S; | 1.58 |
| | | L108H; L219I | |
| t899762 | 179 | T102E; L104M; F107H | 1.57 |
| t899556 | 180 | L104I; F107H; L108T; | 1.52 |
| | | L219I; M222L | |
| t902721 | 181 | F107H; L108M; M222V | 1.48 |
| t899350 | 182 | T102K; F107S; L108H; | 1.48 |
| | | G218S; L219I | |
| t903282 | 183 | T102H; F107S; L108H; | 1.42 |
| | | G218A; M222V | |
| t899988 | 184 | T102E; L104I; F107H; | 1.40 |
| | | M222V | |
| t900028 | 185 | T102H; F107H; L108T; | 1.33 |
| | | G218A; L219I; M222L | |
| t902760 | 186 | L104M; L108V; L219I; | 1.28 |
| | | M222T | |
| t903294 | 187 | T102K; L104M; L108H; | 1.26 |
| | | G218A; M222L | |
| t899423 | 188 | T102H; L108H; G218S | 1.24 |
| t899758 | 189 | T102E; F107Y; L108H; | 1.20 |
| | | G218S; L219I | |
| t900185 | 190 | T102E; F107Y; L108T; | 1.18 |
| | | G218A; M222L | |
| t903098 | 191 | F107H; L108H; G218A | 1.18 |
| t902752 | 192 | T102E; L104M; F107Y; | 1.12 |
| | | L108T; G218S; M222I | |
| t902836 | 193 | L104M; F107Y; L108V; | 1.06 |
| | | G218S; L219I; M222L | |
| t903866 | 194 | T102S; F107S; L108Q; | 1.03 |
| | | L219I; M222L | |
| t903316 | 195 | T102S; F107S; L108H; | 1.03 |
| | | L219I; M222N | |

TABLE 5
Sequences of ALs described in Example 1 and Example 2

| Strain ID | Amino Acid SEQ ID NO: | Nucleotide SEQ ID NO: |
|---|---|---|
| t888841 | 1 | 2 |
| t888850 | 3 | 196 |
| t915919 | 4 | 197 |
| t900097 | 5 | 198 |
| t900270 | 6 | 199 |
| t900424 | 7 | 200 |
| t902826 | 8 | 201 |
| t900166 | 9 | 202 |
| t903111 | 10 | 203 |
| t903984 | 11 | 204 |
| t899644 | 12 | 205 |
| t903141 | 13 | 206 |
| t899646 | 14 | 207 |
| t900622 | 15 | 208 |
| t904262 | 16 | 209 |
| t899973 | 17 | 210 |
| t900177 | 18 | 211 |
| t902547 | 19 | 212 |
| t899454 | 20 | 213 |
| t900320 | 21 | 214 |
| t904103 | 22 | 215 |
| t900578 | 23 | 216 |
| t899285 | 24 | 217 |
| t903447 | 25 | 218 |
| t902439 | 26 | 219 |
| t904333 | 27 | 220 |
| t903300 | 28 | 221 |
| t900309 | 29 | 222 |
| t904359 | 30 | 223 |
| t899349 | 31 | 224 |
| t904360 | 32 | 225 |
| t900541 | 33 | 226 |
| t903485 | 34 | 227 |
| t903905 | 35 | 228 |
| t900376 | 36 | 229 |
| t900070 | 37 | 230 |
| t903327 | 38 | 231 |
| t903331 | 39 | 232 |
| t899517 | 40 | 233 |
| t902928 | 41 | 234 |
| t903870 | 42 | 235 |
| t900570 | 43 | 236 |
| t901073 | 44 | 237 |
| t900842 | 45 | 238 |
| t903554 | 46 | 239 |
| t902525 | 47 | 240 |
| t900314 | 48 | 241 |
| t903000 | 49 | 242 |
| t900137 | 50 | 243 |
| t902777 | 51 | 244 |
| t900470 | 52 | 245 |
| t903261 | 53 | 246 |
| t899659 | 54 | 247 |
| t903839 | 55 | 248 |
| t903683 | 56 | 249 |
| t903786 | 57 | 250 |
| t900534 | 58 | 251 |
| t899681 | 59 | 252 |
| t899385 | 60 | 253 |
| t899234 | 61 | 254 |
| t903684 | 62 | 255 |
| t902742 | 63 | 256 |
| t901124 | 64 | 257 |
| t903556 | 65 | 258 |
| t900261 | 66 | 259 |
| t903832 | 67 | 260 |
| t899661 | 68 | 261 |
| t900441 | 69 | 262 |
| t904233 | 70 | 263 |
| t902516 | 71 | 264 |
| t900250 | 72 | 265 |
| t902538 | 73 | 266 |
| t900135 | 74 | 267 |
| t904309 | 75 | 268 |
| t899303 | 76 | 269 |
| t903599 | 77 | 270 |
| t900257 | 78 | 271 |
| t900204 | 79 | 272 |
| t903569 | 80 | 273 |
| t903678 | 81 | 274 |
| t900518 | 82 | 275 |
| t899485 | 83 | 276 |
| t903247 | 84 | 277 |
| t902757 | 85 | 278 |
| t902788 | 86 | 279 |
| t899939 | 87 | 280 |
| t903070 | 88 | 281 |
| t901084 | 89 | 282 |
| t900075 | 90 | 283 |
| t899817 | 91 | 284 |
| t900304 | 92 | 285 |
| t903671 | 93 | 286 |
| t899801 | 94 | 287 |
| t903810 | 95 | 288 |
| t903609 | 96 | 289 |
| t904224 | 97 | 290 |
| t899897 | 98 | 291 |
| t904382 | 99 | 292 |
| t899614 | 100 | 293 |
| t900493 | 101 | 294 |
| t900056 | 102 | 295 |
| t902703 | 103 | 296 |
| t903478 | 104 | 297 |
| t903415 | 105 | 298 |
| t900280 | 106 | 299 |
| t904001 | 107 | 300 |
| t897903 | 108 | 301 |
| t900237 | 109 | 302 |
| t903852 | 110 | 303 |
| t903783 | 111 | 304 |
| t902507 | 112 | 305 |
| t903472 | 113 | 306 |
| t903445 | 114 | 307 |
| t899874 | 115 | 308 |
| t903669 | 116 | 309 |
| t899582 | 117 | 310 |
| t903313 | 118 | 311 |
| t903018 | 119 | 312 |
| t904043 | 120 | 313 |
| t899778 | 121 | 314 |
| t902749 | 122 | 315 |
| t903837 | 123 | 316 |
| t899595 | 124 | 317 |
| t903999 | 125 | 318 |
| t899254 | 126 | 319 |
| t899587 | 127 | 320 |
| t903126 | 128 | 321 |
| t897909 | 129 | 322 |
| t899316 | 130 | 323 |
| t903927 | 131 | 324 |
| t899276 | 132 | 325 |
| t903921 | 133 | 326 |
| t899696 | 134 | 327 |
| t899835 | 135 | 328 |
| t900002 | 136 | 329 |
| t897927 | 137 | 330 |
| t902820 | 138 | 331 |
| t899604 | 139 | 332 |
| t899513 | 140 | 333 |
| t903230 | 141 | 334 |
| t900395 | 142 | 335 |
| t904192 | 143 | 336 |
| t899840 | 144 | 337 |
| t904358 | 145 | 338 |
| t899871 | 146 | 339 |
| t902832 | 147 | 340 |
| t899261 | 148 | 341 |
| t903895 | 149 | 342 |
| t902834 | 150 | 343 |
| t899266 | 151 | 344 |
| t902888 | 152 | 345 |
| t903908 | 153 | 346 |
| t899507 | 154 | 347 |
| t900251 | 155 | 348 |
| t903988 | 156 | 349 |
| t902587 | 157 | 350 |
| t900125 | 158 | 351 |
| t900247 | 159 | 352 |
| t899718 | 160 | 353 |
| t899788 | 161 | 354 |
| t900451 | 162 | 355 |
| t899501 | 163 | 356 |
| t897929 | 164 | 357 |
| t904107 | 165 | 358 |
| t900001 | 166 | 359 |
| t899786 | 167 | 360 |
| t902438 | 168 | 361 |
| t902554 | 169 | 362 |
| t902740 | 170 | 363 |
| t900090 | 171 | 364 |
| t902435 | 172 | 365 |
| t904324 | 173 | 366 |
| t903946 | 174 | 367 |
| t900007 | 175 | 368 |
| t897910 | 176 | 369 |
| t897898 | 177 | 370 |
| t900300 | 178 | 371 |
| t899762 | 179 | 372 |
| t899556 | 180 | 373 |
| t902721 | 181 | 374 |
| t899350 | 182 | 375 |
| t903282 | 183 | 376 |
| t899988 | 184 | 377 |
| t900028 | 185 | 378 |
| t902760 | 186 | 379 |
| t903294 | 187 | 380 |
| t899423 | 188 | 381 |
| t899758 | 189 | 382 |
| t900185 | 190 | 383 |
| t903098 | 191 | 384 |
| t902752 | 192 | 385 |
| t902836 | 193 | 386 |
| t903866 | 194 | 387 |
| t903316 | 195 | 388 |

It should be appreciated that sequences disclosed in this application may or may not contain secretion signals. The sequences disclosed in this application encompass versions with or without secretion signals. It should also be understood that amino acid sequences disclosed in this application may be depicted with or without a start codon (M). The sequences disclosed in this application encompass versions with or without start codons. Accordingly, in some instances amino acid numbering may correspond to amino acid sequences containing secretion signal and/or a start codon, while in other instances, amino acid numbering may correspond to amino acid sequences that do not contain a secretion signal and/or a start codon. It should also be understood that sequences disclosed in this application may be depicted with or without a stop codon. The sequences disclosed in this application encompass versions with or without stop codons.

### EQUIVALENTS

Those skilled in the art will recognize or be able to ascertain using no more than routine experimentation, many equivalents to the specific embodiments of the invention described in the present application. Such equivalents are intended to be encompassed by the following claims.

All references, including patent documents, are incorporated by reference in their entirety.

What is claimed is:

1. A host cell that comprises a heterologous polynucleotide encoding an aromatic amino acid ammonia lyase (AL), wherein the amino acid sequence of the AL comprises:
a) a histidine (H) at a position corresponding to position 102 in the sequence of SEQ ID NO: 1;
b) an isoleucine (I) at a position corresponding to position 104 in the sequence of SEQ ID NO: 1;
c) a valine (V) at a position corresponding to position 104 in the sequence of SEQ ID NO: 1;
d) a histidine (H) at a position corresponding to position 107 in the sequence of SEQ ID NO: 1;
e) a serine(S) at a position corresponding to position 107 in the sequence of SEQ ID NO: 1;
f) a tyrosine (Y) at a position corresponding to position 107 in the sequence of SEQ ID NO: 1;
g) a methionine (M) at a position corresponding to position 108 in the sequence of SEQ ID NO: 1; or
h) any combination thereof.

2. The host cell of claim 1, wherein the AL is a phenylalanine ammonia lyase (PAL).

3. The host cell of claim 2, wherein the amino acid sequence of the PAL comprises substitutions at positions corresponding to the following positions in the sequence of SEQ ID NO: 1:
i. positions 102, 104, and 218;
ii. positions 104, 108, and 218;
iii. positions 102, 104, 108, 218, and 222;
iv. positions 102 and 222;
v. positions 102, 104, and 219;
vi. positions 102, 108, and 222;
vii. positions 102, 108, 218, and 222;
viii. positions 102 and 218;
ix. positions 102, 104, 108, and 222;
x. positions 102, 104, and 108;
xi. positions 102, 218, and 222;
xii positions 102, 104, 219, and 222;
xiii. positions 102 and 108;
xiv. positions 104 and 222;
xv. positions 102, 108, and 218; or
xvi. positions 104 and 108.

4. The host cell of either one of claim 2 or 3, wherein the amino acid sequence of the PAL comprises the following amino acid substitutions relative to the sequence of SEQ ID NO: 1:
i. T102H, L104M, and G218A;
ii. L104M, L108T, and G218A;
iii. T102E, L104M, L108T, G218A, and M222L;
iv. T102S and M222L;
v. T102H, L104M, and L219I;
vi. T102H, L104M, L108T, G218A, and M222V;
vii. T102S, L108T, and M222L;
viii. T102S, L108T, G218S, and M222L;
ix. T102E, L108T, and M222I;
x. T102E and G218S;
xi. T102K, L104I, L108T, and M222L;
xii. T102S, L104M, and L108M;
xiii. T102K, G218A, and M222T;
xiv. T102S, L104M, L219I, and M222L;
xv. T102H and L108T;
xvi. L104M and M222V;
xvii. T102H, L104M, G218A, and M222T;
xviii. T102S, L108V, and G218A;
xix. L104A, L108T, and G218A;
xx. L104V and L108T; or
xxi. T102K, L108V, and M222L.

5. The host cell of any one of claims 2-4, wherein the amino acid sequence of the PAL comprises the following amino acid substitutions relative to the sequence of SEQ ID NO: 1:
i. T102H, L104A, and G218A;
ii. T102K, L104V, L219I, and M222V;
iii. T102K, L108V, and M222L;
iv. T102H, L108M, G218A, and M222T;
v. T102K, L104A, and M222I;
vi. T102K and M222T;
vii. T102K and L104I;
viii. L104M and M222V;
ix. T102S, L108M, and G218S;
x. T102E and L108M;
xi. T102E, L108M, and G218A;
xii. T102S and L108M;
xiii. L102K and L108M; or
xiv. L108M.

6. The host cell of claim 1, wherein the AL is a tyrosine ammonia lyase (TAL).

7. The host cell of claim 6, wherein the amino acid sequence of the TAL comprises substitutions at positions corresponding to the following positions in the sequence of SEQ ID NO: 1:
i. positions 104, 108, 219, and 222;
ii. positions 102, 108, 218, and 219;
iii. positions 102, 104, 108, 219, and 222;
iv. positions 102, 107, 108, 218, 219, and 222;
v. positions 104, 108, 218, 219, and 222;
vi. positions 102, 104, 107, and 222;
vii. positions 102, 104, 107, 108, 219, and 222;
viii positions 104, 218, and 222;
ix. positions 102, 108, 218, 219, and 222;
x. positions 104, 108, and 218;
xi. positions 102, 107, 108, 219, and 222;
xii. positions 104, 107, 108, and 222;
xiii positions 102, 104, 108, 218, and 219;
xiv. positions 102, 104, 107, 219, and 222;
xv. positions 102, 108, 218, and 222;
xvi. positions 102, 108, and 222;
xvii. positions 102, 104, 108, and 219;
xviii. positions 102, 104, 107, 108, 218, 219, and 222;
xix. positions 102, 104, 107, 108, 218, and 219;
xx. positions 102, 107, 108, 219, and 222;
xxi. positions 102, 104, 107, 108, 218, and 222; or
xxii. positions 102, 104, 107, 108, and 219.

8. The host cell of either one of claim 6 or 7, wherein the amino acid sequence of the TAL comprises the following amino acid substitutions relative to the sequence of SEQ ID NO: 1:
i. L104A, L108Q, L219I, and M222N;
ii. T102S, L108Q, G218A, and L219I;
iii. T102H, L104M, L108M, L219I, and M222L;
iv. T102E, F107Y, L108M, G218S, L219I, and M222N;
v. L104I, L108H, G218A, L219I, and M222V;
vi. T102E, L104M, F107Y, and M222I;
vii. T102E, L104V, F107Y, L108M, L219I, and M222T;
viii. T102S, L104I, G218S, L219I, and M222V;
ix. L104V, G218A, and M222L;
x. T102K, L108H, G218A, L219I, and M222T;
xi. L104I, L108M, and G218S;
xii. T102H, F107Y, L108M, L219I, and M222V;
xiii. L104V, F107H, L108Q, and M222L;
xiv. T102K, L104A, L108Q, G218A, and L219I;
xv. T102S, L104A, F107S, L219I, and M222N;
xvi. T102S, L108H, G218S, and M222V;
xvii. T102K, L104A, L108H, L219I, and M222N;
xviii. T102S, L108H, and M222N;
xix. T102H, L104M, L108M, and L219I;
xx. T102K, L104A, F107Y, L108V, G218A, L219I, and M222N;
xxi. T102H, L108M, G218S, and M222L;
xxii. T102E, L104M, F107Y, L108M, G218A, and L219I;
xxiii. T102E, L104V, F107H, and M222N;
xxiv. T102H, F107H, L108M, L219I, and M222T;
xxv. T102H, L104V, F107S, L108Q, G218S, and M222T;
xxvi. T102E, L104M, F107S, L108M, G218A, and L219I; or
xxvii. T102E, L104V, F107Y, L108M, and L219I.

9. A host cell that comprises a heterologous polynucleotide encoding an aromatic amino acid ammonia lyase (AL), wherein the amino acid sequence of the AL comprises an amino acid substitution at a position corresponding to amino acid residue F107 relative to the sequence of SEQ ID NO: 1.

10. The host cell of claim 9, wherein the amino acid sequence of the AL comprises:
a) a histidine (H) at a position corresponding to position 107 in the sequence of SEQ ID NO: 1;
b) a serine(S) at a position corresponding to position 107 in the sequence of SEQ ID NO: 1; or
c) a tyrosine (Y) at a position corresponding to position 107 in the sequence of SEQ ID NO: 1.

11. A host cell that comprises:
a first heterologous polynucleotide encoding an aromatic amino acid ammonia lyase (AL), wherein the amino acid sequence of the AL comprises one or more amino acid substitutions relative to the sequence of SEQ ID NO:1, and
a second heterologous polynucleotide encoding a coumarate ligase (4CL).

12. A mixture comprising:
a) a host cell comprising a first heterologous polynucleotide encoding an aromatic amino acid ammonia lyase (AL), wherein the amino acid sequence of the AL comprises one or more amino acid substitutions relative to the sequence of SEQ ID NO: 1, and
b) a medium comprising exogenously supplied glucose, phosphoenolpyruvate, erythrose 4-phosphate, 3-deoxy-D-arabino-hept-2-ulosonate 7-phosphate, 3-dehydroquinate, 3-dehydroshikimate, shikimate, chorismate, prephenate, phenylpyruvate, hydroxyphenylpyruvate, phenylalanine, or tyrosine.

13. The host cell or mixture of any one of claims 9-12, wherein the amino acid sequence of the AL comprises an amino acid substitution at a position corresponding to amino acid residue 102, 104, 107, 108, 218, 219, or 222 relative to the sequence of SEQ ID NO: 1.

14. The host cell or mixture of any one of claims 11-13, wherein the AL comprises:
i. a serine(S) at a position corresponding to position 102 in the sequence of SEQ ID NO: 1;
ii. a glutamic acid (E) at a position corresponding to position 102 in the sequence of SEQ ID NO: 1;
iii. a lysine (K) at a position corresponding to position 102 in the sequence of SEQ ID NO: 1;
iv. a histidine (H) at a position corresponding to position 102 in the sequence of SEQ ID NO: 1;
v. a methionine (M) at a position corresponding to position 104 in the sequence of SEQ ID NO: 1;
vi. an alanine (A) at a position corresponding to position 104 in the sequence of SEQ ID NO: 1;
vii. an isoleucine (I) at a position corresponding to position 104 in the sequence of SEQ ID NO: 1;
viii. a valine (V) at a position corresponding to position 104 in the sequence of SEQ ID NO: 1;
ix. a histidine (H) at a position corresponding to position 107 in the sequence of SEQ ID NO: 1;
x. a serine(S) at a position corresponding to position 107 in the sequence of SEQ ID NO: 1;
xi. a tyrosine (Y) at a position corresponding to position 107 in the sequence of SEQ ID NO: 1;
xii. a threonine (T) at a position corresponding to position 108 in the sequence of SEQ ID NO: 1;
xiii. a valine (V) at a position corresponding to position 108 in the sequence of SEQ ID NO: 1;
xiv. a glutamine (Q) at a position corresponding to position 108 in the sequence of SEQ ID NO: 1;
xv. a methionine (M) at a position corresponding to position 108 in the sequence of SEQ ID NO: 1;
xvi. an alanine (A) at a position corresponding to position 218 in the sequence of SEQ ID NO: 1;
xvii. a serine(S) at a position corresponding to position 218 in the sequence of SEQ ID NO: 1;
xviii. an isoleucine (I) at a position corresponding to position 219 in the sequence of SEQ ID NO: 1;
xix. a leucine (L) at a position corresponding to position 222 in the sequence of SEQ ID NO: 1;
xx. an asparagine (N) at a position corresponding to position 222 in the sequence of SEQ ID NO: 1;
xxi. an isoleucine (I) at a position corresponding to position 222 in the sequence of SEQ ID NO: 1;
xxii. a valine (V) at a position corresponding to position 222 in the sequence of SEQ ID NO: 1;
xxiii. a threonine (T) at a position corresponding to position 222 in the sequence of SEQ ID NO: 1; or
xxiv. any combination thereof.

15. The host cell or mixture of any one of claims 11-14, wherein the AL is a phenylalanine ammonia lyase (PAL).

16. The host cell or mixture of claim 15, wherein relative to the sequence of SEQ ID NO: 1, the PAL comprises substitutions at positions corresponding to the following positions in the sequence of SEQ ID NO: 1:
i. positions 102, 104, and 218;
ii. positions 104, 108, and 218;
iii. positions 102, 104, 108, 218, and 222;
iv. positions 102 and 222;
v. positions 102, 104, and 219;
vi. positions 102, 108, and 222;
vii. positions 102, 108, 218, and 222;
viii positions 102 and 218;
ix. positions 102, 104, 108, and 222;
x. positions 102, 104, and 108;
xi. positions 102, 218, and 222;
xii. positions 102, 104, 219, and 222;
xiii. positions 102 and 108;
xiv. positions 104 and 222;
xv. positions 102, 108, and 218; or
xvi. positions 104 and 108.

17. The host cell or mixture of either one of claim 15 or 16, wherein the amino acid sequence of the PAL comprises the following amino acid substitutions relative to the sequence of SEQ ID NO: 1:
i. T102H, L104M, and G218A;
ii. L104M, L108T, and G218A;
iii. T102E, L104M, L108T, G218A, and M222L;
iv. T102S and M222L;
v. T102H, L104M, and L219I;
vi. T102H, L104M, L108T, G218A, and M222V;
vii. T102K and G218A;
viii. T102S, L108T, and M222L;
ix. T102S, L108T, G218S, and M222L;
x. T102E, L108T, and M222I;
xi. T102E and G218S;
xii. T102K, L104I, L108T, and M222L;
xiii. T102S, L104M, and L108M;
xiv. T102K, G218A, and M222T;
xv. T102S, L104M, L219I, and M222L;
xvi. T102H and L108T;
xvii. L104M and M222V;
xviii. T102H, L104M, G218A, and M222T;
xix. T102S, L108V, and G218A;
xx. L104A, L108T, and G218A;
xxi. L104V and L108T; or
xxii. T102K, L108V, and M222L.

18. The host cell or mixture of any one of claims 11-14, wherein the AL is a tyrosine ammonia lyase (TAL).

19. The host cell or mixture of claim 18, wherein the TAL comprises substitutions at positions corresponding to the following positions in the sequence of SEQ ID NO: 1:
i. positions 104, 108, 219, and 222;
ii. positions 102, 108, 218, and 219;
iii. positions 102, 104, 108, 219, and 222;
iv. positions 102, 107, 108, 218, 219, and 222;
v. positions 104, 108, 218, 219, and 222;
vi. positions 102, 104, 107, and 222;
vii. positions 102, 104, 107, 108, 219, and 222;
viii. positions 104, 218, and 222;
ix. positions 102, 108, 218, 219, and 222;
x. positions 104, 108, and 218;
xi. positions 102, 107, 108, 219, and 222;
xii positions 104, 107, 108, and 222;
xiii. positions 102, 104, 108, 218, and 219;
xiv. positions 102, 104, 107, 219, and 222;
xv. positions 102, 108, 218, and 222;
xvi positions 102, 108, and 222;
xvii. positions 102, 104, 108, and 219;
xviii. positions 102, 104, 107, 108, 218, 219, and 222;
xix. positions 102, 104, 107, 108, 218, and 219;
xx. positions 102, 107, 108, 219, and 222;
xxi positions 102, 104, 107, 108, 218, and 222; or
xxii. positions 102, 104, 107, 108, and 219.

20. The host cell or mixture of either one of claim 18 or 19, wherein the amino acid sequence of the TAL comprises the following amino acid substitutions relative to the sequence of SEQ ID NO: 1:
i. L104A, L108Q, L219I, and M222N;
ii. T102S, L108Q, G218A, and L219I;
iii. T102H, L104M, L108M, L219I, and M222L;
iv. T102E, F107Y, L108M, G218S, L219I, and M222N;
v. L104I, L108H, G218A, L219I, and M222V;
vi. T102E, L104M, F107Y, and M222I;
vii. T102E, L104V, F107Y, L108M, L219I, and M222T;
viii. T102S, L104I, G218S, L219I, and M222V;
ix. L104V, G218A, and M222L;
x. T102K, L108H, G218A, L219I, and M222T;
xi. L104I, L108M, and G218S;
xii. T102H, F107Y, L108M, L219I, and M222V;
xiii. L104V, F107H, L108Q, and M222L;
xiv. T102K, L104A, L108Q, G218A, and L219I;
xv. T102S, L104A, F107S, L219I, and M222N;
xvi T102S, L108H, G218S, and M222V;
xvii. T102K, L104A, L108H, L219I, and M222N;
xviii. T102S, L108H, and M222N;
xix. T102H, L104M, L108M, and L219I;
xx. T102K, L104A, F107Y, L108V, G218A, L219I, and M222N;
xxi. T102H, L108M, G218S, and M222L;
xxii. T102E, L104M, F107Y, L108M, G218A, and L219I;
xxiii. T102E, L104V, F107H, and M222N;
xxiv. T102H, F107H, L108M, L219I, and M222T;
xxv. T102H, L104V, F107S, L108Q, G218S, and M222T;
xxvi. T102E, L104M, F107S, L108M, G218A, and L219I; or
xxvii. T102E, L104V, F107Y, L108M, and L219I.

21. The host cell or mixture of any one of claims 18-20, wherein the amino acid sequence of the TAL comprises the following amino acid substitutions relative to the sequence of SEQ ID NO: 1:
i. T102E, L104V, F107Y, and L108H;
ii. T102E, F107Y, L108H, G218A, and M222I;
iii. T102S, F107Y, L108H, G218A, and M222T;
iv. T102E, L104M, F107Y, L108H, and G218A;
v. L219I and M222T;
vi. F107Y, L108H, L219I, and M222T;
vii. L104A, L108Q, L219I, and M222N;
viii. T102S, L108Q, G218A, and L219I;
ix. T102H, L104M, L108M, and L219I;
x. M222L;
xi. T102E, F107Y, L108M, and G218S;
xii. L219I and M222N;
xiii. L104I, L108H, G218A, and L219I;
xiv. M222V;
xv. T102E, L104M, F107Y, and M222I;
xvi. T102E, F107Y, L108H, and M222I;
xvii. T102E, F107Y, L108H, and G218A;
xviii. T102S, F107Y, and L108H;
xix. T102E, F107Y, L108H, and M222T; or
xx. T102E, F107Y, L108H, and L219I.

22. The host cell of any of claims 1-21, wherein the AL comprises an amino acid sequence that has at least 90% identity to the sequence of SEQ ID NO: 1.

23. The host cell of any of claims 1-22, wherein the heterologous polynucleotide comprises a sequence that is at least 90% identical to the sequence of SEQ ID NO: 2.

24. The host cell of any one of claims 1-23, wherein the host cell is a bacterial cell, an archaebacterial cell, an algal cell, a fungal cell, a yeast cell, a plant cell, an animal cell, a mammalian cell, or a human cell.

25. The host cell of claim 24, wherein the host cell is a filamentous fungi cell or a yeast cell.

26. The host cell of claim 25, wherein the yeast cell is a Saccharomyces cell, a Yarrowia cell, a Komagataella cell, or a Pichia cell.

27. The host cell of claim 26, wherein the Saccharomyces cell is a Saccharomyces cerevisiae cell.

28. The host cell of claim 25, wherein the yeast cell is Yarrowia cell.

29. The host cell of claim 24, wherein the host cell is a bacterial cell.

30. The host cell of claim 29, wherein the bacterial cell is an E. coli cell.

31. The host cell of any one of claims 1-30, wherein the AL is able to convert phenylalanine to trans-cinnamic acid.

32. The host cell of any one of claims 1-31, wherein the AL is able to convert tyrosine to p-coumaric acid.

33. The host cell of any one of claims 1-32, comprising one or more enzymes of the shikimate pathway capable of converting phosphoenolpyruvate and erythrose 4-phosphate to chorismate.

34. The host cell of any one of claims 1-33, wherein one or more of the enzymes of the shikimate pathway are encoded by a heterologous polynucleotide.

35. The host cell of any one of claims 1-34, wherein the amino acid sequence(s) of one or more of the enzymes of the shikimate pathway comprise one or more substitutions relative to the amino acid sequence(s) of a wild-type shikimate pathway enzyme.

36. The host cell of any one of claims 1-35, further comprising a heterologous polynucleotide encoding a cinnamate 4-hydroxylase (C4H), a heterologous polynucleotide encoding a coumarate ligase (4CL), or both.

37. The host cell of claim 36, wherein the amino acid sequence of C4H comprises one or more substitutions relative to the amino acid sequence of a parent C4H (SEQ ID NO: 389).

38. The host cell of claim 36, wherein the amino acid sequence of 4CL comprises one or more substitutions relative to the amino acid sequence of wild-type 4CL.

39. The host cell of any one of claims 1-38, further comprising a heterologous polynucleotide encoding one, two, three, four, five, or all of: a coumarate ligase (4CL), a double bond reductase (DBR), a chalcone synthase (CHS), a chalcone 3-hydroxylase (CH₃H), an O-methyltransferase (OMT), and an UDP dependent glycosyltransferase (UGT).

40. The host cell of claim 39, wherein the amino acid sequence(s) of one, two, three, four, five, or all of 4CL, DBR, CHS, CH3H, OMT, or UGT comprises one or more substitutions relative to the amino acid sequence(s) of a wild-type version of the protein.

41. An aromatic amino acid ammonia lyase (AL), wherein the amino acid sequence of the AL comprises:
a) a histidine (H) at a position corresponding to position 102 in the sequence of SEQ ID NO: 1;
b) an isoleucine (I) at a position corresponding to position 104 in the sequence of SEQ ID NO: 1;
c) a valine (V) at a position corresponding to position 104 in the sequence of SEQ ID NO: 1;
d) a histidine (H) at a position corresponding to position 107 in the sequence of SEQ ID NO: 1;
e) a serine(S) at a position corresponding to position 107 in the sequence of SEQ ID NO: 1;
f) a tyrosine (Y) at a position corresponding to position 107 in the sequence of SEQ ID NO: 1;
g) a methionine (M) at a position corresponding to position 108 in the sequence of SEQ ID NO: 1; or
h) any combination thereof.

42. The AL of claim 41, wherein the AL is a phenylalanine ammonia lyase (PAL).

43. The AL of claim 42, wherein the amino acid sequence of the AL comprises substitutions at positions corresponding to the following positions in the sequence of SEQ ID NO: 1:
i. positions 102, 104, and 218;
ii. positions 104, 108, and 218;
iii. positions 102, 104, 108, 218, and 222;
iv. positions 102 and 222;
v. positions 102, 104, and 219;
vi. positions 102, 108, and 222;
vii. positions 102, 108, 218, and 222;
viii positions 102 and 218;
ix. positions 102, 104, 108, and 222;
x. positions 102, 104, and 108;
xi. positions 102, 218, and 222;
xii. positions 102, 104, 219, and 222;
xiii. positions 102 and 108;
xiv. positions 104 and 222;
xv. positions 102, 108, and 218; or
xvi. positions 104 and 108.

44. The AL of either one of claim 41 or 43, wherein the amino acid sequence of the AL comprises the following amino acid substitutions relative to the sequence of SEQ ID NO: 1:
i. T102H, L104M, and G218A;
ii. L104M, L108T, and G218A;
iii. T102E, L104M, L108T, G218A, and M222L;
iv. T102S and M222L;
v. T102H, L104M, and L219I;
vi. T102H, L104M, L108T, G218A, and M222V;
vii. T102S, L108T, and M222L;
viii. T102S, L108T, G218S, and M222L;
ix. T102E, L108T, and M222I;
x. T102E and G218S;
xi. T102K, L104I, L108T, and M222L;
xii. T102S, L104M, and L108M;
xiii. T102K, G218A, and M222T;
xiv. T102S, L104M, L219I, and M222L;
xv. T102H and L108T;
xvi. L104M and M222V;
xvii. T102H, L104M, G218A, and M222T;
xviii. T102S, L108V, and G218A;
xix. L104A, L108T, and G218A;
xx. L104V and L108T; or
xxi. T102K, L108V, and M222L.

45. The AL of any one of claims 41-44, wherein the amino acid sequence of the AL comprises the following amino acid substitutions relative to the sequence of SEQ ID NO: 1:
i. T102H, L104A, and G218A;
ii. T102K, L104V, L219I, and M222V;
iii. T102K, L108V, and M222L;
iv. T102H, L108M, G218A, and M222T;
v. T102K, L104A, and M222I;
vi. T102K and M222T;
vii. T102K and L104I;
viii. L104M and M222V;
ix. T102S, L108M, and G218S;
x. T102E and L108M;
xi. T102E, L108M, and G218A;
xii. T102S and L108M;
xiii. L102K and L108M; or
xiv. L108M.

46. The AL of claim 41, wherein the AL is a tyrosine ammonia lyase (TAL).

47. The AL of claim 41, wherein the amino acid sequence of the AL comprises substitutions at positions corresponding to the following positions in the sequence of SEQ ID NO: 1:
i. positions 104, 108, 219, and 222;
ii. positions 102, 108, 218, and 219;
iii. positions 102, 104, 108, 219, and 222;
iv. positions 102, 107, 108, 218, 219, and 222;
v. positions 104, 108, 218, 219, and 222;
vi. positions 102, 104, 107, and 222;
vii. positions 102, 104, 107, 108, 219, and 222;
viii. positions 104, 218, and 222;
ix. positions 102, 108, 218, 219, and 222;
x. positions 104, 108, and 218;
xi. positions 102, 107, 108, 219, and 222;
xii. positions 104, 107, 108, and 222;
xiii. positions 102, 104, 108, 218, and 219;
xiv positions 102, 104, 107, 219, and 222;
xv. positions 102, 108, 218, and 222;
xvi. positions 102, 108, and 222;
xvii. positions 102, 104, 108, and 219;
xviii. positions 102, 104, 107, 108, 218, 219, and 222;
xix. positions 102, 104, 107, 108, 218, and 219;
xx. positions 102, 107, 108, 219, and 222;
xxi. positions 102, 104, 107, 108, 218, and 222; or
xxii. positions 102, 104, 107, 108, and 219.

48. The AL of either one of claim 41 or 47, wherein the amino acid sequence of the AL comprises the following amino acid substitutions relative to the sequence of SEQ ID NO: 1:
i. L104A, L108Q, L219I, and M222N;
ii. T102S, L108Q, G218A, and L219I;
iii. T102H, L104M, L108M, L219I, and M222L;
iv. T102E, F107Y, L108M, G218S, L219I, and M222N;
v. L104I, L108H, G218A, L219I, and M222V;
vi. T102E, L104M, F107Y, and M222I;
vii. T102E, L104V, F107Y, L108M, L219I, and M222T;
viii. T102S, L104I, G218S, L219I, and M222V;
ix. L104V, G218A, and M222L;
x. T102K, L108H, G218A, L219I, and M222T;
xi. L104I, L108M, and G218S;
xii. T102H, F107Y, L108M, L219I, and M222V;
xiii. L104V, F107H, L108Q, and M222L;
xiv. T102K, L104A, L108Q, G218A, and L219I;
xv. T102S, L104A, F107S, L219I, and M222N;
xvi. T102S, L108H, G218S, and M222V;
xvii. T102K, L104A, L108H, L219I, and M222N;
xviii. T102S, L108H, and M222N;
xix. T102H, L104M, L108M, and L219I;
xx. T102K, L104A, F107Y, L108V, G218A, L219I, and M222N;
xxi. T102H, L108M, G218S, and M222L;
xxii. T102E, L104M, F107Y, L108M, G218A, and L219I;
xxiii. T102E, L104V, F107H, and M222N;
xxiv. T102H, F107H, L108M, L219I, and M222T;
xxv. T102H, L104V, F107S, L108Q, G218S, and M222T;
xxvi. T102E, L104M, F107S, L108M, G218A, and L219I; or
xxvii. T102E, L104V, F107Y, L108M, and L219I.

49. The AL of any of claims 41-48, wherein the amino acid sequence of the AL comprises an amino acid sequence that has at least 90% identity to the sequence of SEQ ID NO: 1.

50. An aromatic amino acid ammonia lyase (AL), wherein the amino acid sequence of the AL comprises an amino acid substitution at a position corresponding to amino acid residue F107 relative to the sequence of SEQ ID NO: 1.

51. The AL of claim 50, wherein the amino acid sequence of the AL comprises:
a) a histidine (H) at a position corresponding to position 107 in the sequence of SEQ ID NO: 1;
b) a serine(S) at a position corresponding to position 107 in the sequence of SEQ ID NO: 1;
c) a tyrosine (Y) at a position corresponding to position 107 in the sequence of SEQ ID NO: 1.

52. The AL of either one of claim 50 or 51, wherein the amino acid sequence of the AL comprises an amino acid substitution at a position corresponding to amino acid residue 102, 104, 108, 218, 219, or 222 relative to the sequence of SEQ ID NO: 1.

53. The AL of any one of claims 41-52, wherein the AL produces more trans-cinnamic acid per unit time than an AL with an amino acid sequence comprising the sequence of SEQ ID NO: 1.

54. The AL of any one of claims 41-53, wherein the AL can produce at least 10%, 20%, 30%, 40%, 50%, 60%, 70%, 80%, 90%, 100%, 200%, or 300% more trans-cinnamic acid per unit time than a AL with an amino acid sequence comprising the sequence of SEQ ID NO: 1.

55. The AL of any one of claims 41-54, wherein the AL can produce at least 10%, 20%, 30%, 40%, 50%, 60%, 70%, 80%, 90%, 100%, 200%, or 300% more trans-cinnamic acid per unit time than coumarate per unit time.

56. The AL of any one of claims 46-52, wherein the AL produces more coumarate per unit time than a TAL with an amino acid sequence comprising the sequence of SEQ ID NO: 1.

57. The AL of any one of claim 46-52 or 56, wherein the AL can produce at least 10%, 20%, 30%, 40%, 50%, 60%, 70%, 80%, 90%, 100%, 200%, or 300% more coumarate per unit time than a TAL with an amino acid sequence comprising the sequence of SEQ ID NO: 1.

58. The AL of any one of claim 46-52, or 56-57, wherein the AL can produce at least 10%, 20%, 30%, 40%, 50%, 60%, 70%, 80%, 90%, 100%, 200%, or 300% more coumarate per unit time than trans-cinnamic acid per unit time.

59. A method of producing an aromatic compound, comprising contacting phenylalanine and/or tyrosine with a host cell of any one of claims 1-40 or an AL of any one of claims 41-58.

60. The method of claim 59, comprising contacting phenylalanine.

61. The method of claim 59 or 60, comprising contacting tyrosine.

62. The method of any one of claims 59-61, wherein the aromatic compound is a flavor or fragrance compound.

63. The method of any one of claims 59-62, wherein the aromatic compound is a phenylpropanoid.

64. The method of any one of claims 59-63, wherein the aromatic compound is a sweetener.

65. The method of any one of claims 59-64, wherein the aromatic compound is a flavonoid.

66. The method of any one of claims 59-64, wherein the aromatic compound is a flavanone.

67. The method of any one of claim 59-64 or 66, wherein the aromatic compound is eriodictyol or a glycoside and/or alkoxy derivative thereof.

68. The method of any one of claim 59-64 or 66, wherein the aromatic compound is hesperetin.

69. The method of any one of claims 59-63, wherein the aromatic compound is a dihydrochalcone.

70. The method of any one of claim 59-64 or 69, wherein the aromatic compound is hesperetin dihydrochalcone 4′-O-glucoside (HDG).

71. The method of any one of claims 59-62, wherein the aromatic compound is vanillin.

72. The method of any one of claims 59-63, wherein the aromatic compound is an hydroxycinnamic acid or a derivative thereof.

73. The method of claim 72, wherein the hydroxycinnamic acid or the derivative thereof is coumaric acid, ferulic acid, sinapic acid, caffeic acid, chlorogenic acid, or rosmarinic acid.

74. The method of 73, wherein the aromatic compound is ferulic acid.

75. A method of improving an aromatic compound manufacturing mixture, comprising contacting the mixture with the AL of any one of claims 41-58.

76. The method of claim 75, wherein the method is a method of improving a flavor or fragrance manufacturing mixture.

77. The method of claim 75 or 76, wherein the aromatic compound manufacturing mixture comprises a shikimate pathway product.

78. The method of claim 77, wherein the shikimate pathway product comprises: chorismate, prephenate, phenylpyruvate, hydroxyphenylpyruvate, phenylalanine, or tyrosine.

79. The method of any one of claims 76-78, wherein improving comprises converting phenylalanine to trans-cinnamic acid.

80. The method of any one of claims 76-78, wherein improving comprises converting tyrosine to coumarate.

81. The method of any one of claims 76-80, wherein improving comprises promoting production of an aromatic compound.

82. The method of any one of claims 59-81, wherein the method occurs in vitro.
